(19) United States
(12) Patent Application Publication
(10) Pub. No.: US 2021/0390262 A1
(43) Pub. Date: Dec. 16, 2021

(54) STANDARDIZED DATA INPUT FROM LANGUAGE USING UNIVERSAL SIGNIFICANCE CODES

(71) Applicants: Mette Dyhrberg, Brooklyn, NY (US); Christopher V. Beckman, Los Angeles, CA (US)

(72) Inventors: Mette Dyhrberg, Brooklyn, NY (US); Christopher V. Beckman, Los Angeles, CA (US)

(21) Appl. No.: 17/344,884

(22) Filed: Jun. 10, 2021

Related U.S. Application Data
(60) Provisional application No. 63/037,551, filed on Jun. 10, 2020.

(51) Int. Cl. G06F 40/30; G16H 10/60; G16H 70/00; G16H 50/70

(52) U.S. Cl. CPC G06F 40/30; G16H 10/60; G16H 40/67; G16H 50/70; G16H 70/00

(57) ABSTRACT

New systems, devices, methods and other techniques for Digital Health and Digital Therapeutics are provided. In some embodiments, new techniques are provided for creating and managing personal health-related data and generating Digital Therapeutics interventions based thereon, using a new, specialized computer hardware and software control system. In some embodiments, new graphical user interfaces (“GUIs”) are provided for generating, storing and managing personal health-related data and Digital Health and Digital Therapeutics based thereon. In some embodiments, health-related data is normalized based on translation vectors. In some embodiments, standardized significance codes are defined and implemented, based on the usage of terms by user(s) of the control system. Subsequent entry of such terms during data entry by a user then causes the control system to enter data at least partially defined by such significance codes.

### CROSS-REFERENCE TO RELATED APPLICATIONS

This application claims the benefit of U.S. Provisional Application No. 63/037,551, filed Jun. 10, 2020, titled “Standardized Data Input from Language Using Universal Significance Codes,” which is herein incorporated by reference in its entirety.

### TECHNICAL FIELD

The present invention relates to the field of systems, devices and methods for gathering and managing health-related data and delivering Digital Therapeutics, and, in particular, for standardizing such data with the aid of user interfaces.

### BACKGROUND

Personal digital assistants (“PDAs”) are small portable computers that allow a user to record and manage personal information, and they have been available in some form for decades. For example, as early as the 1970s, small digital wristwatches allowed users to perform personal computing, such as financial arithmetic, and storing information related to personal contacts, such as names, addresses and phone numbers. The now virtually ubiquitous smartphones can be thought of as modern PDAs, capable of sophisticated, highly secure communications over a network, and running some of the most complex computer programs. Specialized software designed to be run on smartphones, known as “Apps,” allow users to provide and receive a wide variety of data, and perform a wide variety of functions based on those data, ranging from online banking to digital gaming.

Some such Apps relate to personal health and/or fitness management (a.k.a., “Health and Fitness” Apps. For example, at least some such Apps, and some other software, are known as “Digital Health” software, which, as used in the present application, means software: aiding a user(s) (and/or their caregivers, and/or friends and family) in managing the user's(s') health- and/or fitness-related: i. behavior; ii. environment; and/or iii. information. Some such Digital Health software is used with associated hardware, such as a heart rate monitor, blood pressure sensor, or other health-related sensors and actuators. Some Digital Health software and hardware falls within the definition of “Digital Therapeutics.”

As used in the present application, “Digital Therapeutics” means: evidence-based therapeutic interventions, driven by software, to: a) prevent, manage and/or treat an adverse and/or unwanted physical, mental and/or behavioral illness, disorder and/or condition; and/or b) create a beneficial and/or desired physical, mental and/or behavioral illness, disorder and/or condition.

Some Health and Fitness Apps, and some Digital Health Apps, may be “Telehealth” software, meaning that the App enables a doctor or other caregiver to provide a remote examination of and/or consultation to a user (e.g., a patient). Similarly, some Health and Fitness Apps, and some Digital Health Apps, may be software related to “Adherence,” meaning that the software enables a user and/or their caregiver(s) to monitor and aid the user in maintaining a regimen of pharmaceutical(s) or other nutrient(s), environmental factor(s) or behavioral intervention(s) in accordance with a plan.

Turning next to the general development of Scientific Method, scientific experiments have been conducted at least for several centuries. The Scientific Method is an observation-based approach to developing scientific theories, which are generally accepted propositions of fact based on repeated, validated testing. In some formulations of the Scientific Method, scientists brainstorm testable hypotheses based on their current knowledge and intuition, emphasizing hypotheses that can generate predictions, which, if testable and untrue, would result in disproving those hypotheses (assuming that sufficiently rigorous testing methods are employed). If such rigorous testing fails to disprove such a hypothesis, and additional testing by different scientists similarly fails to disprove the hypothesis, a generally accepted theory may emerge over time, increasing the body of scientific knowledge. Generally speaking, conjecture, without repeated, validated testing, by itself, is not scientific knowledge—even if it is based on well-known, carefully observed correlations, sound logic and/or a widely-held consensus. Nonetheless, many people even today, and in centuries past, have turned to such conjecture in managing their personal affairs, including their health.

It should be noted that some of the disclosures set forth as background, such as, but not limited to, the above language under the heading “Background,” may not relate exclusively to prior art and the state of the art in the field(s) of the invention, and should not be construed as an admission with respect thereto.

### SUMMARY

New systems, devices, methods and other techniques for Digital Health and/or Digital Therapeutics are provided. In some embodiments, new techniques are provided for creating and managing personal health-related data and generating interventions based thereon. In some embodiments, new graphical user interfaces (“GUIs”) and sub-tools thereof are provided which, in conjunction with one or more peripheral devices, generate personal health-related data, and enhance user control over health-related activities and factors through unique dynamic interplay with users and their caregivers. In some embodiments, such GUIs and sub-tools are provided with the aid of specialized hardware and software including, and/or included within, a control system. In some such embodiments, a dynamic array of user-adjustable GUI sub-tools alter their prominence (e.g., by location, size, sub-features and/or effects) both in reaction to, and to prioritize, user attention and actions. In some embodiments, such alterations in prominence are based on health-related data points, user behaviors, environmental variables, and/or dynamic relationships between them. In some embodiments, such personal health-related data is generated by user input and/or peripheral devices.

In some embodiments, instigations related to user actions are selected and prioritized based on health-related data via a control system carrying out in-body experiments. In some such embodiments, hypotheses based on correlations between personal health data variables are generated and ranked based on a probability of validity, then tested retroactively, and drive interventions by the control system. In some embodiments, Digital Therapeutics are generated without hypotheses, or, instead, with multiple or incomplete hypotheses. In some embodiments, instigated health-related data is time-varied, varied in different combinations, and reassessed in comparison to changing health statuses in real time. In some embodiments, hypotheses or combinations of data and therapeutics are generated or re-ranked, based on such reassessments, and the process repeats.

In some embodiments, retroactive and/or mock experiments are run and managed by a control system, based on robust, wide-ranging data gathering related to states of health and related activities and factors correlated with, impacting or otherwise linked to those states of health.

In some embodiments, health-related data is normalized based through the use of Translation Vectors, incorporating Significance Maps.

As mentioned above, the techniques may include methods and systems, in some embodiments. In some embodiments, such systems include computer hardware and software, including non-transitory machine-readable media with executable instructions. When executed by computer hardware, the instructions may cause the systems to carry out any or all of the methods set forth in this application.

These and other aspects of the invention will be made clearer below, in other parts of this application. This Summary, the Abstract, and other parts of the application, are for ease of understanding only, and no part of this application should be read to limit the scope of any other part, nor to limit the scope of the invention, whether or not it references matter in any other part.

Further aspects of the invention will be set forth in greater detail, below, with reference to the particular figures.

### BRIEF DESCRIPTION OF THE DRAWINGS

The features and advantages of the example embodiments of the invention presented herein will become more apparent from the detailed description set forth below when taken in conjunction with the following drawings.

FIG. 1 is a front view of an example Digital Therapeutics user with an example smartphone implementing example Digital Therapeutics techniques, at the outset of a time period for delivering such Digital Therapeutics, in accordance with some embodiments.

FIG. 2 is a front view of the same example Digital Therapeutics user, with the same example smartphone implementing additional example Digital Therapeutics techniques involving an additional example peripheral device, at a later time in the day, in accordance with some embodiments.

FIG. 3 is a front view of the same example Digital Therapeutics user, with the same example smartphone implementing additional example Digital Therapeutics techniques, at a later time in the day than that pictured in FIGS. 1 and 2, above, including some example dynamic Digital Therapeutics tools in accordance with some embodiments.

FIG. 4 is a front view of the same example Digital Therapeutics user, with the same example smartphone implementing example alternative embodiments of Digital Therapeutics techniques, at the same time of day pictured in FIG. 3, above, in accordance with some embodiments.

FIG. 5 is a front view of the same example Digital Therapeutics user, with the same example smartphone implementing example alternative embodiments of Digital Therapeutics techniques, at the same time of day pictured in FIGS. 3 and 4, above, in accordance with some embodiments.

FIG. 6 is a front view of the same example Digital Therapeutics user, with the same example smartphone implementing some example alternative embodiments of Digital Therapeutics techniques, at the same time of day pictured in FIGS. 3, 4 and 5, above.

FIG. 7 is an enlargement, for magnification purposes, of a view pictured in FIG. 5, depicting aspects of an example tracking indicator including example new, different sub-tools (in comparison to other forms of the tracking indicator).

FIG. 8 depicts the same general form of tracking indicator pictured previously, in FIG. 7, under different circumstances, and has a decreased Digital Therapeutics deficit than that pictured in FIG. 7, and some additional example aspects of sub-tools, in accordance with some embodiments.

FIG. 9 depicts the same general form of tracking indicator as pictured previously, in FIGS. 7 and 8, at an earlier point in time, with the user having achieved all goal data and/or actions required for that point in time.

FIG. 10 is a schematic block diagram of some example elements of an example control system that may be used to implement various aspects of the present invention, some of which aspects are described in reference to FIGS. 1-9 and 11-16 of this application, in accordance with some embodiments.

FIG. 11 is a process flow diagram, setting forth several example steps that may be undertaken by a control system (such as the example control system set forth above, in reference to FIG. 10) implementing some aspects of the present invention, according to some embodiments.

FIG. 12 is a front view of an example GUI, implementing some example aspects of the present invention related to monitoring and gathering data related to a user (e.g., personal health data and behavior), in accordance with some embodiments.

FIG. 13 is a perspective view of an example environment in the process of being monitored by an example imaging sensor, which may be controlled by a control system including computer hardware and software (such as any of the control systems set forth in this application), in accordance with some embodiments.

FIG. 14 is a front view of an example user interface incorporating graphical aspects, which display personal health-related data over time for a user of hardware and software aspects set forth in the present application.

FIG. 15 is a front view of the same example user interface, now shown in an alternate format, at a later time, with altered tools and sub-tools, and some additional sub-tools, in accordance with some embodiments.

FIG. 16 is a process flow diagram, setting forth several example steps that may be undertaken by a control system (such as the example control system set forth above, in reference to FIG. 10) implementing some aspects of the present invention, according to some embodiments.

It should be noted that the figures referenced above are examples only of the wide variety of different embodiments falling within the scope of the invention, as will be readily apparent to those skilled in the art. Thus, any particular size(s), shape(s), proportion(s), scale(s), material(s) or number(s) of elements pictured are illustrative and demonstrative, and do not limit the scope of invention, as will be so readily apparent.

### DETAILED DESCRIPTION

The example embodiments of the invention presented herein are directed to systems, devices and methods for managing health with new, specialized information technology, including Digital Health and Digital Therapeutics, which systems, devices and methods are now described herein. This description is not intended to limit the application of the example embodiments presented herein. In fact, after reading the following description, it will be apparent to one skilled in the relevant art(s) how to implement the following example embodiments in alternative embodiments.

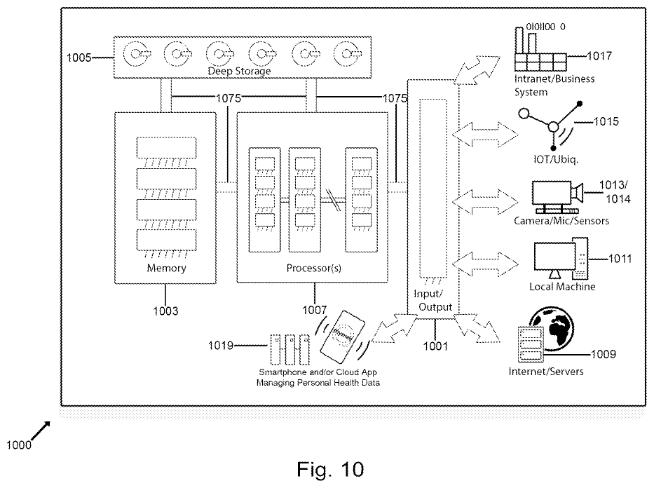

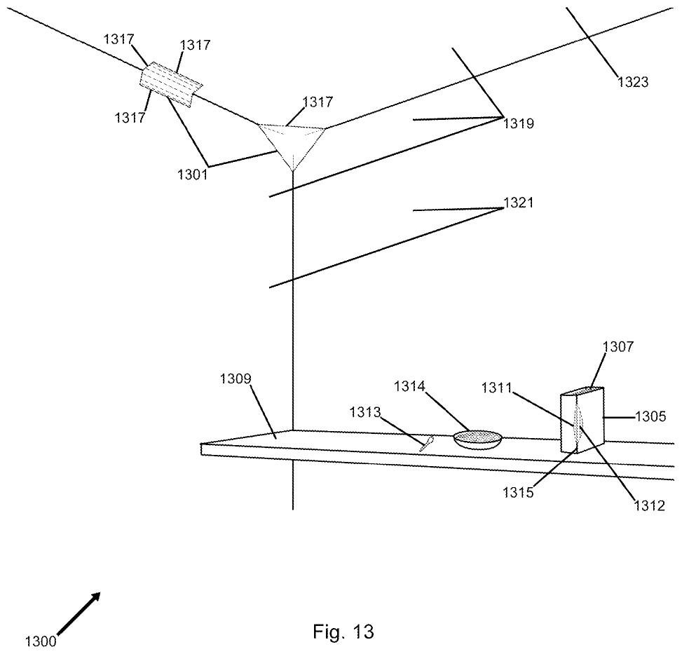

FIG. 1 is a front view of an example Digital Therapeutics user 100 holding a personal computing device (a “PCD”), namely, example smartphone 101, implementing example Digital Therapeutics techniques at the outset of a time period (e.g., a day in the life of the user) for delivering Digital Therapeutics, in accordance with some embodiments. At the outset, it is important to note that, although the example of a smartphone 101 is provided, any of the techniques set forth herein may be practiced, instead or in addition, with other forms of PCDs and other devices comprising, or comprised within, computer hardware, such as the example computer hardware set forth below as control system 1000, in FIG. 10. For example, in some embodiments, a user may be holding or otherwise interacting with another form of personal electronics comprising and/or comprised within such a control system, such as a personal digital assistant device (“PDA”), desktop computer and/or external peripheral devices which, in some embodiments, are not handheld (e.g., a wall-, ceiling- or otherwise environmentally-mounted display device, and/or any number of ambient intelligence, augmented reality, mixed reality and/or display devices), any of which may carry out each of the techniques set forth herein with respect to smartphone 101, in various embodiments. Some examples of such devices are provided below, for example, in reference to FIGS. 10 and 13.

Regardless of the form of computing device used, in some embodiments, the computer hardware and software of the control system may create a user interface, such the example shown as graphical user interface (“GUI”) 103 for gathering, accessing, storing and processing health- and fitness-related information, for executing Digital Therapeutics techniques based on that information, and delivering particular Digital Therapeutics to a user (e.g., example user 100). As just some examples, some such information includes, but is not limited to: biometrics; vital signs; genomic information; proteomic information; genotype information; phenotype information; biomarkers; exercise-related information; activity-related information; environmental information; dietary information; drug and/or drug treatment adherence information; other adherence-related information; and behavioral information. In some embodiments, such as that pictured, such a user interface 103 is included and presented on a graphical display, such as example smartphone display 105. In some embodiments, smartphone display 105 may present several standard-shaped health data, user behavior, or other health-related aspect tracking tools, such as example tracking indicators 107. Example tracking indicators 107 (and the other similar tracking indicators, shown in a grid totaling twelve (12) tracking indicators, in the example pictured) each present complex health-related data points within GUI sub-tools. In some embodiments, such health-related data points are based on user behavior and status, at particular points in time. In the example instance pictured, user 100 may have just awoken, picked up smartphone 101, and activated a software application controlling and creating GUI 103. Being at the outset of the time period in which health-related data will be gathered and Digital Therapeutics will be administered, no such newly-gathered data for the time period is yet reflected in GUI 103. Instead, GUI 103 only reflects activities, data and goals for the user 100 that have been planned or suggested for the user during the time period, as one or more Digital Therapeutics. As a result, the data points related to unachieved goals for the user 100 are presented within GUI sub-tools—for example, unachieved goal data points are presented within example unachieved data point sub-tools 109, within each example tracking indicator (or, in some embodiments, within another GUI tool).

It should also be noted that, at the outset of the time period, each of tracking indicators 107 have the same, or a substantially similar, overall size and shape (e.g., the identical rounded square overall size and shape pictured for example tracking indicators 107), in some embodiments. Also at the outset of the time period, each of tracking indicators 107 have standard locations (e.g., within a grid layout 111) in some embodiments. However, as will be explained in greater detail below, in some embodiments, such size(s), shape(s) and location(s) are altered over time and/or as a result of user activities and tracking of health-related data, in some embodiments. In some such embodiments, such size(s), shape(s) and location(s) may be based on the relative proportion or urgency of achieving particular therapeutic and data tracking goals for a particular user. In some such embodiments, such urgency is based on a data point(s) based on an algorithm with a weighting for (i.e., altering the algorithm's numeric value(s) output based on) data related to a particular user activities and goals. In some embodiments, such an algorithm is based on a weighting of percentages of goals achieved. In some embodiments, such an algorithm is based on a weighting of a rate of achievement of such percentages of goals. In some embodiments, such an algorithm is based on a weighting of a consistency of achievement of such rates and/or percentages of such goals.

In some embodiments, each of unachieved data point sub-tools 109 may present a number of instances or units of a particular therapeutic activity which are an advised goal for the user 100 to achieve. In some such embodiments, such unachieved data point sub-tools 109 are presented in a location, or with an augmentation (e.g., a visual augmentation) indicating that the represented unachieved goal data point is not yet achieved—such as the example null or “0” indicators 113, shown surrounding particular unachieved goal data points within example tracking indicators. Such unachieved data point sub-tools may be presented in particular designated areas on or about any or all tracking indicators of GUI 103, in various embodiments. For example, in some embodiments pictured, such null or “0” indicators are presented at the upper-right-hand corner of each tracking indicator. In some embodiments, some such an augmentation is or includes a non-visual augmentation or effect. For example, in some embodiments, such a non-visual augmentation is a haptic effect (e.g., shaking or buzzing of smartphone 101), accompanying the user's viewing (e.g., determined via tracking the user's eyes as they point at) one of the unachieved data point sub-tools 109. In some embodiments, some such an augmentation is or includes an auditory augmentation or effect. For example, in some embodiments, such an auditory augmentation or effect is a sound effect emanating from, or simulating emanation from, the location of the unachieved data point sub-tools 109.

Achieved goals may similarly be shown in GUI sub-tools within designated areas on or about such tracking indicators, or with an augmentation which indicates to the user 100 that indicated data, and related therapeutic activity, has been achieved. For example, in some embodiments, achieved data point sub-tools, such as the examples shown as achieved data point sub-tools 115, may be provided on or about an area below unachieved data point sub-tools, and without such a null or “0” indicator surrounding them. In some embodiments, a distinctive, other form of augmentation may be used, such as example abutting check mark 116, indicating that the indicated data, and related therapeutic activity, has been achieved.

Each of the tracking indicators may include a number of other GUI sub-tools, as an alternative to, or in addition to, the examples discussed above. Examples of such alternative or additional sub-tools will be discussed in greater detail, below, in reference to a specific example tracking indicator. Some GUI sub-tools may only be created and presented based upon the occurrence of particular user activities and data gathering, in some embodiments. In some embodiments, some GUI sub-tools may alter their size, appearance, location and/or features (e.g., sub-features) upon such occurrence(s). At least some of such alterations are changes in prominence of both the GUI sub-tools and the GUI tools of which they are part, in some embodiments. Examples of such alterations will also be discussed in greater detail below, in the context of an example functional overview of an example GUI tool and sub-tool within it.

One such example tracking indicator, example tracking indicator 117, presents data points related to water consumption by Digital Therapeutics user 100. Because the time period has just begun, as discussed above, example tracking indicator 117 has an overall size and shape (in the instance pictured, a square with rounded corners) substantially matching the size and shape of all other tracking indicators displayed within grid layout 111. Because, as with other tracking indicators within grid layout 111, tracking indicator 117 relates to one type of health-related activity (namely, water consumption) it contains an activity type indicator as a GUI sub-tool, indicating the activity to be tracked with the aid of tracking indicator 117—namely, an example water or hydration subject matter indicator 119, in the form of an image of a fluid-filled cup 120 covered with condensation droplets, such as the example shown as 121. However, in some embodiments, oral, written, or other subject matter indicators may be used, such as example written indicator 122 (which reads “WATER.”) Tracking indicator 117 also includes an unachieved data point sub-tool 123, which may be of the general nature set forth above for example unachieved data point sub-tools 109. Accordingly, unachieved data point sub-tool 123 is depicted as stating a goal data point of “8,” related to an unachieved water consumption goal for the user 100. Furthermore, a unit indicator GUI sub-tool, namely, unit indicator 125, indicates that numbers presented within any GUI sub-tools, such as unachieved data point sub-tool 123, should be understood to be in those units (in this instance, in cups of water, shown abbreviated as “C”). Thus, user 100 should understand the goal data point of “8,” stated in unachieved data point sub-tool 123 to signify that the user has a goal of consuming eight (8) cups of water, to maintain adequate hydration, throughout the daytime period. Similarly, an achieved data point sub-tool 127 may be included within tracking indicator 117, presenting a number (“0”) representing achieved data points based on user activity and status (i.e., cups of water that have been consumed by user 100 during the current time period during which health-related data and therapeutic activities are being tracked).

In some embodiments, a user, such as user 100, may manually enter data using tracking indicator 117. For example, in some embodiments, tracking indicator 117 is user-actuable (e.g., by touching it with her or his thumb or finger, such as example user's finger 129). In some embodiments, each instance of a user touching tracking indicator 117 (or, in some embodiments, touching it for a prerequisite amount of time or intensity over time) may cause the GUI 103 to record one unit of the relevant subject matter, as indicated (i.e., 1 cup of water consumed). Immediately after so touching tracking indicator 117, achieved data point sub-tool 127 would then read or otherwise indicate the integer “1,” rather than “0,” indicating that the user has consumed one cup of water during the current time period, and, likewise, unachieved data point sub-tool 123 would then read or otherwise indicate that the integer “7,” rather than “8,” indicating that one less cup of water remains to be consumed as an unachieved goal during the current time period.

However, in some embodiments, user 100 is not required to tap or touch tracking indicator 117 to record each unit of data relevant to water consumption. In some embodiments, as will be discussed in greater detail below, sensors may track user 100's water consumption, and other such activities, and automatically record such activity. In some such embodiments, such a sensor(s) may determine that a cup, or several cups, of water have been consumed by user 100, over a duration of time, and may communicate and record data indicating such to a control system comprising or comprised within smartphone 101. Such a control system may then alter tracking indicator 117 to reflect such data. In some embodiments, such sensors (e.g., optical cameras 131) may be included within a peripheral device, such as example smartwatch 133.

As yet another example, in some embodiments, other GUI tools may be provided within GUI 103 which allow for the bulk entry of multiple data points. For example, accelerated data entry GUI tool 135 is included, in some embodiments. Accelerated data entry GUI tool 135 includes a time-related data entry sub-tool 137. In some embodiments, such a time-related data entry sub-tool is in the form of a slider, such as example timeline slider 139. By placing her or his finger onto timeline slider 139, and, while maintaining finger contact with smartphone display 105, sliding her or his finger laterally, towards a particular hour-indicating number, such as any of the example hour indicators 141, data corresponding with goal data points of the user 100 which were expected to be achieved when those times had been reached, are automatically entered, in some embodiments. Thus, by so actuating accelerated data entry tool 135, the user may rapidly update data recorded and presented in any number of tracking indicators, to match data goals expected to be achieved by particular points in time. Furthermore, in some embodiments, the user may so rapidly update such data recorded and presented in all, or selected tracking indicators, for particular subjects, while entering others manually, using a selection sub-tool 143. In some embodiments, the user may first press selection sub-tool 143, and then press any or all of the tracking indicators, and then, use the timeline slider 139, as discussed above, to so rapidly update entries for the tracking indicators so selected. In some embodiments, a user may rapidly so select multiple or all tracking indicators, to then rapidly update entries for such multiple tracking indicators, using a multiple selection tool, such as select all button 145.

In some embodiments, user 100 may exit the view shown as GUI 103, for example, by pressing exit button 147 or home button 149. In some such embodiments, additional GUI aspects may then be presented, allowing the user to engage in other data gathering, tracking functions and other Digital Therapeutics, as set forth in greater detail below. However, it should be understood that, in some embodiments, GUI 103 is not required to be presented for the software within an application that presents GUI 103, and data gathering, tracking functions and other Digital Therapeutics, to be active.

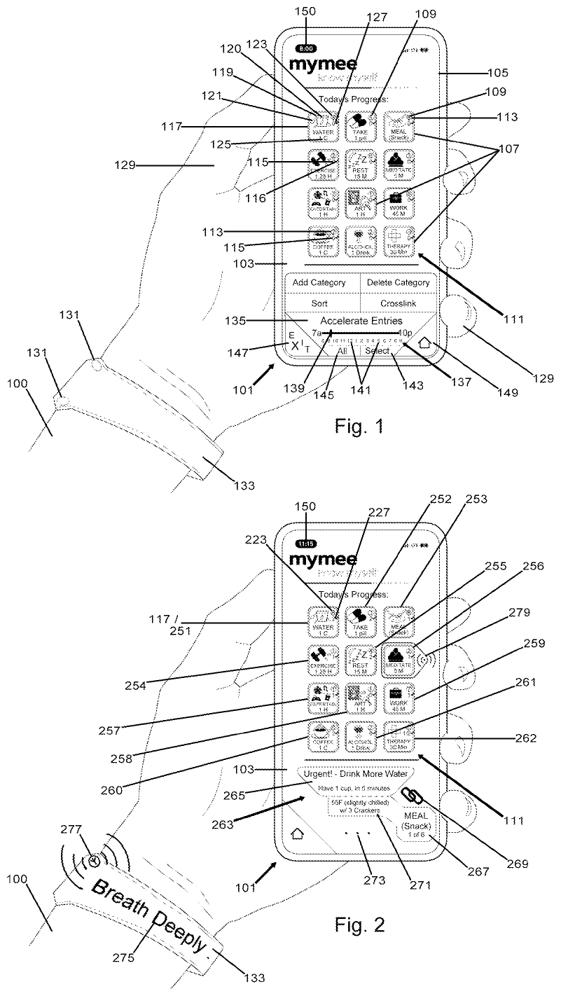

FIG. 2 is a front view of the same example Digital Therapeutics user 100, with the same smartphone 101 implementing additional example Digital Therapeutics techniques involving an additional peripheral device, at a later time in the day, in accordance with some embodiments. Rather than being at the outset of the time period for delivering such Digital Therapeutics, previously shown as the early morning hour (“8:00” am) within GUI clock element 150, several hours have passed, and the relevant point in time depicted is late morning (“11:15” am). In that time, the user 100 has engaged in a number of health-related activities, and has recorded data related thereto using the GUI 103, which is now shown in an updated state reflecting those data. For example, and again returning to example tracking indicator 117, the user has now consumed two (2) cups of water, as now reflected in updated achieved data point sub-tool 227. Corresponding with that change, updated unachieved data point sub-tool 223 now reflects that only six (6) cups of water remain to be consumed in order to reach the goal for water consumption within the current time period for delivering Digital Therapeutics.

As mentioned above, any number of user activities and Digital Therapeutics interventions may be tracked and administered, in various embodiments of the GUI tools and sub-tools and other interventions set forth in the present application. For example, as pictured, a different and visually distinct tracking indicator is provided for each of twelve (12) example subjects within grid layout 111, in some embodiments. In some such embodiments, such subjects and corresponding tracking indicators may include, but are not limited to, the following consumption, activities, environmental factors, cognitive processes and/or other therapeutic subjects: water consumption (tracking indicator 117/251), pharmaceuticals consumption (tracking indicator 252), food consumption (tracking indicator 253), physical exercise (tracking indicator 254), rest or sleep (tracking indicator 255), meditation sessions (tracking indicator 256), entertainment activities (tracking indicator 257), artistic activities (tracking indicator 258), work (tracking indicator 259), coffee consumption (tracking indicator 260), alcohol consumption (tracking indicator 261), and physical or psychological therapy sessions (tracking indicator 262). In some embodiments, certain of those activities, and data related thereto, may be correlated, commonly actuable, or otherwise linked, by new forms of GUI tools, in accordance with some aspects set forth herein.

In the present figure, an expanded user notification and GUI tool area 263 is provided. In some embodiments, as pictured, expanded user notification and GUI tool area 263 may be substantially larger than any of the tracking indicators shown. In this larger format area, larger sub-tools and user messages may appear, in some embodiments, which are easier for the user to view and actuate (e.g., by touch, as discussed above for other GUI tools). For example, an actuable panel, such as example water consumption information panel 265, is provided within GUI tool area 263, in some embodiments. In some embodiments, such a larger sub-tool is provided when a particular level of urgency for a particular activity-related instruction to a user (or other Digital Therapeutic) occurs. For example, in some such embodiments, such a larger sub-tool is provided with respect to a particular activity or instruction to a user of the utmost urgency, over and above all other such activities or instructions. For example, if the user 100 is urgently required to take more water than the data currently entered and tracked indicates, in accordance with an urgency algorithm (as such algorithms are discussed elsewhere in this application), such a large format information panel related to the urgency of water consumption (e.g., example water consumption information panel 265) may be presented within GUI tool area 263. In some embodiments, user 100 may enter data related to the message presented (e.g., register a cup of water drunk) by tapping or touching water consumption information panel 265. Thus, water consumption information panel 265 contains an actuable tool as well as a reminder to the user (namely, that it is “Urgent” for the user to “Drink More Water” as shown). After such data entry, the water consumption information panel 265 may disappear, and the water consumption tracking indicator may be updated to reflect the data entered, as discussed above. In some embodiments, such data entries may still also, or alternatively, be made by directly touching the tracking indicators, not only by actuating expanded format information panels.

Also provided within GUI tool area 263 is a correlated activity GUI tool, in some embodiments—such as actuable food consumption panel 267, which relates to food consumption by the user. As with water consumption information panel 265, food consumption panel 267 may include alerts, instructions or other information relevant to a particular activity (in this instance, food consumption) tracked with GUI 103. Also as with water consumption information panel 265, user 100 may rapidly enter data points relevant to such an activity by touching or tapping anywhere within the actuable area of smartphone display 105 occupied by food consumption panel 267, in some embodiments. In some embodiments, both water consumption information panel 265 and food consumption panel 267 are correlated or otherwise related by a control system comprising, or comprised within, GUI 103—for example, by the control system tracking and recording their common occurrence, or common causality of third factors, in the past, or by a logical rule set within software programming, in various embodiments. In some such embodiments, the common presentation of water consumption information panel 265 and food consumption panel 267 within expanded user notification and GUI tool area 263 is created due to such correlation or other relationships. In some embodiments, actuating either water consumption information panel 265 or food consumption panel 267 will result in the other panel, also, being actuated. In this sense, water consumption information panel 265 and food consumption panel 267 are linked, both logically, and in operation, in some embodiments. To indicate that linked status, a link indicator 269 may be provided, in some embodiments. In some embodiments, an auxiliary actuable bridging and conditioning section 271 may also be provided within food consumption panel 267, and/or within water consumption information panel 265. Bridging and conditioning section 271 may be separately actuable, in some embodiments, and, by touching or tapping on bridging and conditioning section 271, a user may simultaneously actuate water consumption information panel 265 and food consumption panel 267, in some embodiments. In some embodiments, water consumption information panel 265 or food consumption panel 267 may remain independently, and separately actuable. In some embodiments, bridging and conditioning section 271 may also contain information relating to the functional interplay between alerts, instructions or other information relevant to the particular activities so linked. In some embodiments, bridging and conditioning section 271 may include additional sub-tools, to specify an impact of actuating bridging and conditioning section 271, on each type of linked data. One such sub-tool is presented below, as bridging common actuation tool 411. For example, in some embodiments, such a sub-tool is configured to allow a user to specify amounts and/or types of data to be simultaneously entered by touching bridging and conditioning section 271.

In some embodiments, a plurality of such actuable and informational panels may be presented to a user. In some such embodiments, such a plurality of such actuable and informational panels may be presented in a list, from most to least urgency. In some embodiments, only part of that list may appear within GUI 103 at one time. In some such embodiments, a GUI expansion or scrolling tool 273 may be included, allowing a user to view such additional panels within such a list.

As mentioned above, in some embodiments, additional peripheral devices may be comprised within, or comprise, a control system managing and creating user interface 103, and may sense, track and communicate health-related events and activities of user 100 that are sensed to the control system, updating the presentation and function of GUI tools, such as the tracking indicators discussed above. In some embodiments, a wearable peripheral device, such as example smartwatch 133 including such sensors is provided, and worn by user 100 about her or his wrist during a trackable activity. For example, as pictured, user 100 is presently engaged in a meditation activity, tracked and monitored by tracking indicator 256. In some such embodiments, smartwatch 130 may issue GUI-integrated instructions or other Digital Therapeutics to the user, related to the performance of such an activity (and particular qualities thereof). For example, in some embodiments, a user may indicate through the smartwatch that the activity (e.g., a meditation session) has been completed satisfactorily, or the smartwatch may otherwise assess the same (e.g., by sensing sufficiently smooth, rhythmic breathing, or decreased blood pressure, after issuing an instruction 275 to the user, aiding the user in meditation). In any event, however, in some embodiments, after smartwatch 103 so gains such updated health-related events and activities of user 100, it may communicate data representative of those health-related events and activities to the control system managing and creating user interface 103 (e.g., via wireless communications antenna 277). The control system may then update the presentation of tracking indicators (such as tracking indicator 256), in some embodiments. In some embodiments, such communications and updates to presentation due to sharing data between peripheral device(s) and the control system may be termed a “sync” operation. To indicate that such a sync operation is taking place, or has just taken place, a transient indicator may be included, in some embodiments, such as sync indicator 279.

It should be understood, with respect to the present figures and embodiments, and with respect to any other figures and embodiments set forth in this application, that a wide variety of additional and/or alternative forms of smartphone(s), PDA(s), smartwatch(es), other peripheral device(s), control system(s), computer hardware, GUI(s), smartphone(s), and other device(s), system(s) and method(s) and step(s) may be created, used or implemented, in different embodiments of the invention. The exact number, disposition, arrangement, form and direction of GUI elements, tools, and peripheral devices provided herein are only examples of the myriad alternative and additional embodiments falling within the scope of the invention, as will be readily apparent to those of ordinary skill in the art to which the present invention relates.

Similarly, as will be apparent to those of ordinary skill in the art to which the present invention relates, GUI 103, in general, may be formed in a wide variety of alternative shapes, sizes and dimensions, and may track a wide variety of additional, and different user, environmental, 3rd-party, research and other health-related data in various embodiments of the invention. For example, in some embodiments, such a GUI may include behavioral data (e.g., social interactions of the user), the user's heart rate, blood pressure, blood, skin or other bodily material analytes (e.g., via blood-testing hardware), and biomarkers, via similar or different GUI tools, as set forth above. In some such embodiments, the GUI and control system comprised within smartphone 101 may instead be comprised within a form of bodily apparel or a wall-mounted or environmentally embedded computer, with other forms of display elements (e.g., via 3-dimensional (“3D”) display hardware) presented to user 100, instead of, or in addition to, smartphone 101.

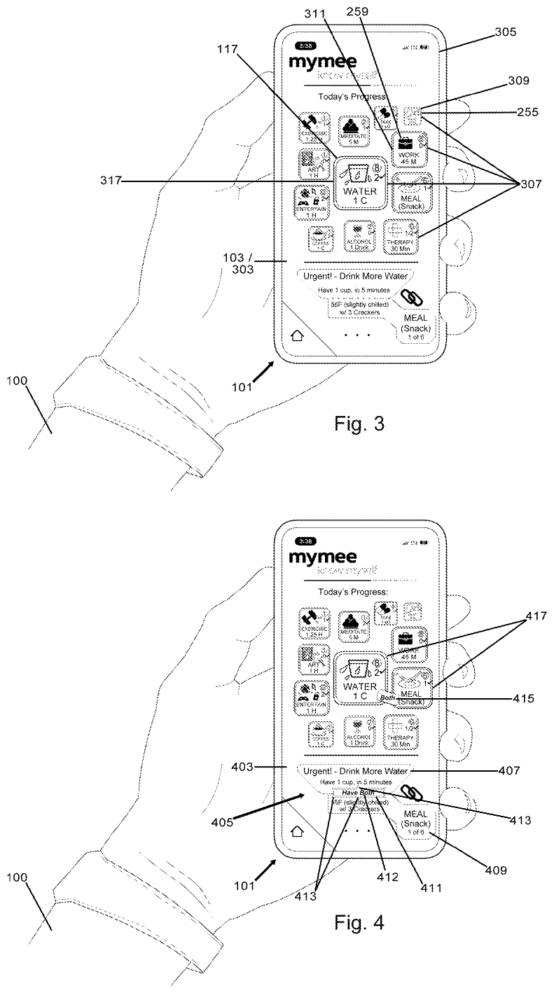

FIG. 3 is a front view of the same example Digital Therapeutics user 100, with the same example smartphone 101 implementing additional example Digital Therapeutics techniques, at a later time in the day than that pictured in FIGS. 1 and 2, above, including some example additional dynamic Digital Therapeutics tools in accordance with some embodiments. At the later point in the day pictured, the GUI, now shown as GUI 303, has an altered layout, based on changed health-related data that has been gathered and presented in Digital Therapeutics techniques administered in the time that has elapsed since the point in time depicted in FIG. 2, above. Among other things, several of the tracking indicators, such as the examples now shown as altered tracking indicators 307 are altered tracking indicators, having an altered appearance based on those changing data over time. For example, in some embodiments, such tracking indicators have migrated in complex patterns, and rearranged their apparent location within the display space of GUI 303, based on those changing data over time, as pictured. For example, whereas tracking indicator 117 was previously (at the time shown in FIG. 2) located in the upper-left-hand corner of GUI 103, it has moved to a location at or about the center of GUI 303, in the position now shown as GUI position 317. In addition, several of those tracking indicators have also changed in apparent size, in some embodiments. In some embodiments, several of those tracking indicators also change in one or more other ways, instead of, or in addition to such location, size or other changes in appearance. In some embodiments, any or all of the above changes, regardless of their type, may be based on an algorithm. In some such embodiments, such algorithms incorporate at least some of such changed health-related data, as will be discussed in greater detail below.

As another example of such changes in appearance, in some embodiments, such tracking indicators change in shape, and such changes in shape are based on such changing data. For example, in some embodiments, such tracking indicators become stellated, or less rounded in appearance, expressing urgency of data and instructions to the user.

In some embodiments, such changes in tracking indicators are accompanied by visual or other effects (e.g., a symbol, filter or other outer or overall graphical augmentation), on, about or otherwise relating to the tracking indicators, which visual or other effects are based on such changing data. In some embodiments, such changes in tracking indicators otherwise change in appearance, based on such changing data.

In some embodiments, such changes in tracking indicators may be accompanied by non-visual indicators and/or other effects. For example, in some embodiments, such tracking indicators may be accompanied by audible sounds or sound effects, which audible sounds or sound effects may be altered based on such changing data. For example, in some embodiments, such audible sounds or sound effects accompany the user's viewing (e.g., determined via tracking the user's eyes as they point at one of the tracking indicators) such a tracking indicator. As another example, in some embodiments, such an auditory augmentation or effect is a sound effect emanating from, or simulating emanation from, the location of such a tracking indicator.

In some embodiments, such tracking indicators may be accompanied by tactile or haptic indicators and/or effects (i.e. “haptic feedback”), which haptic feedback may vary based on such changing data, in some embodiments. In some such embodiments, such haptic feedback may be a vibration and/or a pattern of vibrations. In some embodiments, such haptic feedback may be a tactile simulation of a surface. In some embodiments, such haptic feedback may be in the form of an electronic shock or other charge. In some embodiments, such haptic feedback may accompany the user's interaction with (e.g., touching) such a tracking indicator. As another example, in some embodiments, such an auditory augmentation or effect is an effect emanating from, or simulating emanation from, the location of such a tracking indicator.

In some embodiments, such tracking indicators may be accompanied by olfactory or taste indicators and/or effects (i.e. “olfactory feedback”), which olfactory feedback may vary based on such changing data, in some embodiments. In some such embodiments, such olfactory feedback may be delivered by a scent disbursement actuator. In some such embodiments, such a sense disbursement actuator may combine and spray different amounts of source scent materials (e.g., terpenes), to deliver particular perceived scents associated with Digital Therapeutics, or data or instructions thereof.

In general, any of the changes in appearance, sounds, indicators and effects, and/or additional effects related to a tracking indicator may also relay representations of the changing health-related data that has been gathered and presented by the control system in GUI 303, in some embodiments. Examples of such specific relaying of data will be discussed in greater detail, below. In some embodiments, such changes in appearance, sounds, indicators and effect, and/or additional, accompanying effects may relay aspects of that changing data.

In some embodiments, any of the above indicators or effects may be provided through smartphone 101 to the user, alone (i.e., not accompanying a tracking indicator, other indicator or effect) or in combination with any or all of the tracking indicators, other indicators, or effects set forth above. In some embodiments, any or all of the above tracking indicators, other indicators or effects, regardless of their type, may be so provided, and based on an algorithm. In some such embodiments, the combination selected by the control system may be based on such an algorithm. In some embodiments, such algorithms incorporate at least some of such changed health-related data, as will be discussed in greater detail below.

Regardless of the form of the changed appearance, or other new or changed perceptible effects based on such changing data, such changes or new effects may be based on an algorithm related to the urgency of the Digital Therapeutics measure represented by the tracking indicator subject to such changes or new effects, in some embodiments. In some such embodiments, such an algorithm related to the urgency of the Digital Therapeutics measure represented by the tracking indicator may cause the control system to create such a changed location, appearance, or other new or changed perceptible effect based on the relative urgency of different Digital Therapeutics treatments represented by different tracking indicators. In some embodiments, any of the above such changes or new effects are “changes in prominence” meaning that they alter the user's tendency to notice the tracking indicator or other indicator to which they relate.

For example, returning the example of tracking indicator 117, which is now in the position shown as 317, its changed location may be based on having an utmost urgency, based on such an algorithm related to the urgency of the Digital Therapeutics measure represented it—namely, the indication that the user should consume more water, a particular amount of water, and/or a particular amount of water over a particular amount of time, among other possible data and instructions which may be included in a particular Digital Therapeutics treatment. Based on the user 100's inadequate consumption of water, in an amount during the time period, or at a rate of consumption, that is lower than that required by the Digital Therapeutics being administered by the control system, the control system has triggered such a change in prominence, as an instigation of greater water consumption by the user 100. As another example, tracking indicator 117 has also changed in size, becoming larger relative to its previous size (as shown in FIG. 2) and relative to other tracking indicators'size (and/or an average size of other tracking indicators within GUI 303, in some embodiments). Thus, in some embodiments, a tracking indicator representing therapeutic treatments of the utmost urgency for the user 100 may be presented most prominently (e.g., at the center of the smartphone 101 display screen 305 and/or GUI (now shown as 303) and as the largest tracking indicator of all tracking indicators within the GUI). In some embodiments, prominence may be differently expressed through GUI 303 (e.g., higher vertical position of tracking indicator within the GUI corresponding with greater prominence). Conversely, tracking indicators of a lower urgency based on such an algorithm, such as example tracking indicator 255 shown at location 309, may migrate in an opposing direction, of lower prominence on smartphone 101 display screen 305 (e.g., towards the edge or periphery of the GUI and screen), as pictured. For example, tracking indicator 255, presenting data and instructions of a Digital Therapeutics treatment to manage user 100's amount, rate and/or other qualities of sleep and/or other rest, may be of a lower urgency because the control system has tracked the user's resting behaviors (e.g., through a heart rate monitor, decreased physical movement, detecting REM, breathing rates, and other biometric indicators of sleep or other rest, via sensors on smartphone 101, and/or other peripheral device(s) connected with the control system) and determined that user 100 has engaged in sufficient sleep and/or other rest, in comparison to goal data stored within the control system for that sleep and/or other rest. As a result, the control system has determined that tracking indicator 255 will be so presented, at a position within GUI 303 indicating such a lower urgency. As another example, one or more tracking indicators with an intermediate urgency, based on such data and an urgency algorithm, may be placed by the control system at a location of intermediate prominence (e.g., not as close to the center of the GUI as tracking indicator 117, but not as far off to the periphery of the GUI as tracking indicator 309, as shown by example intermediate tracking indicator location 311 of tracking indicator 259). Similarly, intermediate tracking indicator location 311, and the intermediate size of tracking indicator 259, may reflect a determination that data and user behavior tracked relative to the tracking indicator (namely, work activities of user 100) are insufficient in comparison to goal data, and more insufficient than data relative to other tracking indicators, but not as insufficient as data relative to a tracking indicator of the utmost urgency. As a result, the control system so places tracking indicator 259 at such an intermediate tracking indicator location (and/or, in some embodiments with an intermediate size, and/or other such indicators or effects, as discussed in this application, associated with such an intermediate urgency.) In some embodiments, all tracking indicators are ranked according to such an urgency algorithm, and different indicators or effects are implemented on, about or in relation to them to reflect that rank. For example, in some embodiments, all tracking indicators are differently-sized, from large to small, in accordance with decreasing relative urgency of their corresponding Digital Therapeutics treatments. As another example, in some embodiments, all tracking indicators are differently positioned, from most-to-least-prominent, in accordance with decreasing relative urgency of their corresponding Digital Therapeutics treatments (e.g., interventions, instigations).

As the day progresses, and user 100 conducts more or less of particular behaviors and activities, and encounters health-related events and environments, and health-related data accumulates, any of which may be sensed by the control system, or entered by a user, such a relative rank and prominence of each tracking indicator may change, reflecting such changes in data, in some embodiments. Such embodiments are preferred. In some such embodiments, such changes occur in real time, or nearly so, and such embodiments are especially preferred.

In some embodiments, the changed prominence discussed above, or other changes in or relative to tracking indicators discussed herein, may be based on an algorithm other than an urgency algorithm. For example, in some embodiments, such an algorithm may be based on the control system's determination that certain health-related data is to be instigated, relative to carrying out an in-body experiment, as will discussed in greater detail elsewhere in this application.

FIG. 4 is a front view of the same example Digital Therapeutics user 100, with the same smartphone 101 implementing example alternative embodiments of Digital Therapeutics techniques, at the same time of day pictured in FIG. 3, above, in accordance with some embodiments. As mentioned above, in reference to FIG. 2, in some embodiments, the GUI delivered to user 100 (now shown as GUI 403) may include an expanded user notification and GUI tool area, now shown as expanded user notification and GUI tool area 405. Also as mentioned above in reference to Fig, 2, several enlarged, specialized tools, with more detailed information, instructions and other Digital Therapeutics tools, may be provided in such an expanded user notification and GUI tool area. Thus, as with the examples set forth above in reference to FIG. 2, a number of such other Digital Therapeutics tools are provided in the present figure, including: 1) a water consumption information panel 407, including an actuable tool as well as a reminder to the user (namely, that it is “Urgent” for the user to “drink more water” as shown); and 2) a correlated activity GUI tool—namely, actuable food consumption panel 409, which relates to food consumption by the user. As mentioned above, in some embodiments, such a consumption information panel 407 and food consumption panel 409 may be correlated or otherwise related by a control system comprising, or comprised within, GUI 403—for example, by the control system tracking and recording their common occurrence, or common causality involving third factors, in the past, or by a logical rule set within software programming. In some such embodiments, their common presentation within expanded user notification and GUI tool area 263 is created due to such correlation or other relationships. However, rather than incorporating an auxiliary actuable bridging and conditioning section within either of those two actuable and informational elements, a new form of bridging actuator is provided, to allow the actuation of both of them simultaneously. More specifically, a bridging common actuation tool 411, placed over at least part of both the water consumption information panel 407 and food consumption panel 409. In some embodiments, by touching or tapping bridging common actuation tool 411, a user may simultaneously and automatically actuate both the water consumption information panel 407 and food consumption panel 409. In some embodiments, by tapping or touching another part of the visible area (other than a part covered by the bridging common actuation tool) of either the water consumption information panel 407 or the food consumption panel 409, a user may still actuate only that actuable element, separately. In some embodiments, such a bridging common actuation tool includes arrows or other indicators, such as internal indicators 413, pointing the user to each actuable element that may be so simultaneously and automatically actuated. In some embodiments, such a bridging common actuation tool includes written instructions, symbols or other communications relaying the function of the bridging common actuation tool. For example, in some embodiments, such instructions or symbols, alone or in combination with other indicators, state that both actuable elements (over which the bridging common actuation tool is placed) are subject to automatic actuation (and entry of health-related data) by actuating the bridging common actuation tool. Hence, bridging common actuation tool 411 bears the label “Have Both,” shown as label 412, so indicating that both 1 cup of water consumption, and 1 meal, will be entered upon the actuation of the bridging common actuation tool.

Such a bridging common actuation tool may be provided over other, and any number of, actuable GUI sub-tools, in some embodiments, allowing such a simultaneous actuation of all of such actuable GUI sub-tools, at the user's discretion, in various embodiments. For example, an additional bridging common actuation tool 415 is also provided, hovering above, and covering from view, at least part of two tracking indicators 417. Because those tracking indicators 417, as discussed above for all such tracking indicators, may have an altered, migrating position, and a wide variety of sub-tools, some of which carry data that are not to be occluded, a wide variety of shapes and sizes for bridging common actuation tools may be provided, in various embodiments of the invention, to allow such visible, common actuation properties, without occluding the presentation of data, instructions and/or other Digital Therapeutics treatments. As pictured, for example, bridging common actuation tool 415 has a smaller profile than that pictured for bridging common actuation tool 411, and is presented at a corner-to-corner, non-horizontal angle. In addition, bridging common actuation tool 415 bears a truncated and/or smaller label 419, to accommodate its smaller form factor.

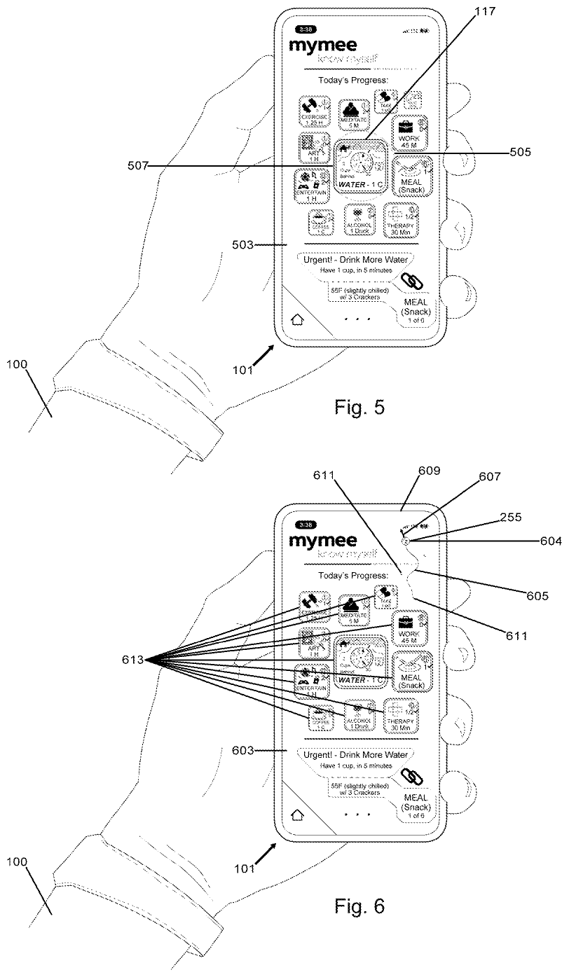

FIG. 5 is a front view of the same example Digital Therapeutics user 100, with the same smartphone 101 implementing some example alternative embodiments of Digital Therapeutics techniques, at the same time of day pictured in FIGS. 3 and 4, above, in accordance with some embodiments. As discussed above, in some embodiments, GUI tools such as tracking indicators may change their appearance based on changing health-related data and user behavior. In some such embodiments, such tracking indicators or other GUI tools may include new, different sub-tools, presented within, on or about particular tracking indicators or other GUI tools, based on such changing health-related data and/or user behavior. As also discussed in greater detail above, in some embodiments, tracking indicators may alter their size and prominence based on an algorithm, such as an urgency algorithm, and based on such changing health-related data and/or user behavior. In some such embodiments, as a tracking indicator or other GUI tool becomes thus enlarged, or otherwise more prominent, reflecting a heightened urgency to administer Digital Therapeutics treatments related to such a GUI tool, such new, different sub-tools are created.

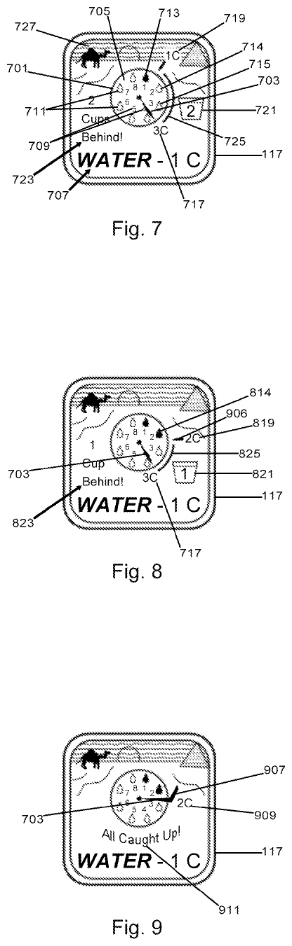

For example, as pictured in the present figure, in some embodiments, as tracking indicator 117 has become enlarged and more prominent, based on such a heightened urgency related to Digital Therapeutics treatments related to it, tracking indicator 117 now includes several such new, different sub-tools within it in the example enlarged and differentiated tracking indicator format 507, each of which new, different sub-tools tracks additional data, and presents data, instructions and/or other Digital Therapeutics measures in more detail than other forms of tracking indicator 117 (e.g., as shown in other figures, and discussed above). Details of such example new, different sub-tools are presented within view 505, which is enlarged for magnification purposes below, in reference to FIG. 7, discussed in greater detail below. Thus, although the smartphone 101 GUI (now shown as GUI 503) includes a layout of tracking indicators generally similar to a GUI form shown previously, in FIGS. 3 and 4 as GUI 303 and GUI 403, tracking indicator exhibits major differences with respect to new sub-tools provided within it. In some embodiments, such new, different sub-tools may accompany any other change in appearance or other effect, or type of such change or other effect set forth in this application. In some embodiments, such accompaniment may be based on such changing data and/or sensed user behavior, as discussed above.

In some such embodiments, as a tracking indicator or other GUI tool becomes thus enlarged, or otherwise more prominent, reflecting a heightened urgency to administer Digital Therapeutics treatments related to such a GUI tool, sub-tools presented within, on or about particular tracking indicators are altered. In some embodiments, such accompaniment may be based on such changing data and/or sensed user behavior, as discussed above. In some embodiments, such alterations to sub-tools may accompany any other change in appearance or other effect, or type of such change or other effect of GUI tools set forth in this application.

FIG. 6 is a front view of the same example Digital Therapeutics user 100, with the same example smartphone 101 implementing some example alternative embodiments of Digital Therapeutics techniques, at the same time of day pictured in FIGS. 3, 4 and 5, above. As discussed above, in some embodiments, some tracking indicators or other GUI tools may be provided with a lower prominence based on an algorithm, health-related data and/or user behavior sensed and implemented by a control system, in some embodiments. For example, as also discussed above, in some embodiments, tracking indicators of a lower urgency based on such an algorithm, such as example tracking indicator 255, may migrate to a position of lower prominence on smartphone 101 display screen 305. In some embodiments, as mentioned above, the control system may have determined that user 100 has engaged in sufficient activities relate to tracking indicator 255, and/or other data indicating that sleep and/or other rest is less urgent at the current time. As also mentioned above, such determinations may be based on a comparison to goal data stored within the control system for that sleep and/or other rest.

In some embodiments, when goal data for the entire current time period (e.g., a day, in the example provided) for a particular tracking indicator has been achieved—e.g., 8 hours of sleep and/or rest, which is goal data reflected previously in the unachieved data point sub-tool of tracking indicator 255—a tracking indicator may substantially shrink (as shown by example shrunken form 604 of tracking indicator 255) and/or disappear entirely from the smartphone 101's GUI (now shown as 603). In some embodiments, such a tracking indicator may move entirely off of the visible part of GUI 603, as shown by an example curving exit path 605 (emulating a bubble floating upward through a fluid medium), and example current movement vector 607 of tracking indicator 255. In a short time following the time pictured in the present figure, tracking indicator may next continue its motion in, or approximately in, the direction of motion shown by movement vector 607, and entirely off of the display screen edge 609. In this sense, such a removal of a Digital Therapeutics GUI tool, after the satisfaction of goals of the related Digital Therapeutics treatment, presents to user 100 as the GUI tool appearing to “floating off” of the GUI 603. In some embodiments, visible traces, such as the example bubble effects 611, of the motion of tracking indicator 255 may linger momentarily (e.g., a few seconds, before fading away) to call attention to the change in the GUI occurring with the exit of tracking indicator 255 off of the display screen 305. Afterwards, GUI 603 is then provided in a simplified, more concentrated collection and presentation of GUI tools, without tracking indicator 255. In some embodiments, at least some of the remaining GUI elements of GUI 603 (e.g., remaining tracking indicators 613) may alter their appearance, after the removal of such another GUI tool. For example, in some embodiments, at least some of remaining tracking indicators 613 may become larger. In some embodiments, tracking indicators 613 may adjust their position to better fill the available display space vacated by tracking indicator 255.

FIG. 7 is an enlargement, for magnification purposes, of view 505 of FIG. 5, discussed above, depicting aspects of an example tracking indicator 117 including example new, different sub-tools (in comparison to other forms of the tracking indicator, discussed above). As discussed above, in some embodiments, as tracking indicator 117 has become enlarged and more prominent, based on heightened urgency, tracking indicator 117 now includes several new, different sub-tools within it in the example enlarged and differentiated tracking indicator format, now shown as 707, each of which new, different sub-tools tracks additional data, and presents data, instructions and/or other Digital Therapeutics measures in more detail than other forms of tracking indicator 117 (e.g., as shown in other figures, and discussed above). Details of such example new, different sub-tools are presented more clearly in the present figure, than in FIG. 5, due to enlargement.

Tracking indicator 117, in format 707, includes several example new sub-tools, provided within it. For example, a new example time- and data-tracking sub-tool 701 is provided. Time- and data-tracking sub-tool 701 may include certain clock-like aspects, such as example hour hand indicator 703. As with known hour hand indicators, hour hand indicator 703 may point in a direction about a clock-like round face 705 of time- and data-tracking sub-tool 701 corresponding with the hour of the day in the user's time zone. Also placed in a circular format, concentric with the overall round shape of time- and data-tracking sub-tool 701, is a circular array of goal data points, such as the examples shown as goal data points 709, each of which goal data points is placed at or near the location corresponding with a point in time during the day when that goal data point is prescribed to be reached by a user. As a result, when hour hand indicator 703 indicates a particular time of day, it also indicates a goal data point, which is to be reached by the user. In addition, in some embodiments, another circular array of achieved goal data point indicators, such as the examples pictured as achieved goal data point indicators 711, may be provided. In some embodiments, each of achieved goal data point indicators 711 may have a different appearance or other indication, based on whether health related data gathered by the control system supports the determination that the user has actually reached the abutting (and corresponding) data point. At the point in time indicated in FIG. 7, the user has consumed one cup of water, which was the goal for the user on or about the first hour and a half for the day. As a result, a goal data point indicator 713 has been filled with color (e.g., blue) so indicating that a first cup of water has been consumed. However, the hour hand indicator 703 is presently at a position corresponding approximately with the 5:00 position of a clock face, indicating that almost 5 hours have elapsed in the time period, and two other goal data points, namely goal data point indicator 714 and goal data point indicator 715, have also been passed by hour hand indicator 703. Because the user has not consumed the two additional cups of water prescribed and indicated by goal data point indicator 714 and goal data point indicator 715, those goal data point indicators remain empty in appearance, without a coloring or shading as shown for goal data point indicator 713.

Thus, using a single time- and data-tracking sub-tool 701, a user can rapidly determine both a Digital Therapeutics goal and achievement, at any point in the current time period subject to Digital Therapeutics techniques. In some embodiments, as with other time-related GUI tools set forth above, the user may touch hour hand indicator 703 in some embodiments, to position it such that it points to a goal data point which has been achieved by the user, and thereby enter corresponding data into the control system.

In some embodiments, additional data point goal and achievement indicators may be used, in addition to, and or as an alternative to those set forth above. For example, in some embodiments, an explicit goal data due indicator 717 is included within time- and data-tracking sub-tool 701. In some embodiments, goal data due indicator 717 sums and indicates the number of Digital Therapeutics actions or data points that were prescribed for the user at the particular time (e.g., the user was required to drink three (3) cups of water by 5:00, which number is thus stated in example goal data due indicator 717). Similarly, in some embodiments, an explicit achieved goal data indicator 719 may be included, which counts and presents the number of Digital Therapeutics actions or data points that were achieved by the user at the current point in time. Thus, in the example pictured, in which the user has consumed one (1) cup of water, achieved goal data indicator 719 reads “1C,” with “C” being an abbreviation for cup, indicating that the user has consumed just one (1) cup of water. In some embodiments, an action or data deficit indicator, such as the example deficit indicator 721, is included in time- and data-tracking sub-tool 701. In some embodiments, the control system assesses a difference between the goal data due (e.g., the number of cups of water due to be consumed) as presented by goal data due indicator 717, and the achieved goal data (e.g., the number of cups actually consumed) as presented by achieved goal data indicator 719, and presents that difference in deficit indicator 721. Thus, to extend the example, deficit indicator 721 indicates that “2” cups of water are due, yet have not been consumed (i.e., the Digital Therapeutics “treatment deficit”), at the relevant point in time shown in the figure. In some embodiments, an even more explicit instruction regarding the Digital Therapeutics treatment deficit may be provided. For example, as pictured, a written or verbal phrase 723 is provided, emphasizing to the user that she or he is “2 cups behind” the goal user action and corresponding data goal for that point in time. In some embodiments, additional demonstrative symbols may otherwise highlight that treatment deficit—such as, emphatic arc 725, shown abutting and scoring the area of time- and data-tracking sub-tool 701 on which the inaction by the user (or other failure to meet goal data) occurred.

In some embodiments, such a deficit may result in more emphatic pervasive sub-tool or effect. For example, in some embodiments, such a more emphatic pervasive sub-tool or effect may be in the form of a GUI area-wide, and/or background-filling effect, such as the example shown as 727, within the smartphone GUI, or a GUI tool, such as time- and data-tracking sub-tool 701. In some embodiments, such an area-wide, and/or background-filling effect may create an atmospheric cue relating to the nature and/or degree of the deficit. Thus, as shown, area-wide, and/or background-filling effect 727 depicts a desert landscape scene, indicating a general atmosphere of a water deficit, to the user. In some embodiments, such an area-wide, and/or background-filling effect may be presented in the event that the Digital Therapeutics treatment deficit exceeds a particular threshold amount (e.g., more than 1 cup of water less), and/or has exceeded a particular threshold too many times in a prior time period (i.e., a particular debt, resulting from such deficits in the past, is exceeded).

Although the example of a clock-like form factor is provided for time- and data-tracking sub-tool 701 is provided, it should be understood that a wide variety of different form of time- and data-tracking sub-tools may be used, alternatively or in addition, in various embodiments of the invention, as will be readily apparent to those of ordinary skill in the art. For example, in some embodiments, a linear format (e.g., a timeline) may be implemented, in an alternate form of time- and data-tracking sub-tool 701.

FIG. 8 depicts the same general form of tracking indicator 117 as pictured previously, in FIG. 7, under different circumstances—namely, wherein a user has consumed one (1) additional cup of water, and thus has a decreased Digital Therapeutics deficit than that pictured in FIG. 7—and some additional example aspects of sub-tools, in accordance with some embodiments. Because the user has consumed one (1) additional cup of water than in the scenario depicted in FIG. 7, an additional goal data point indicator 813 has been filled with color (e.g., blue) or otherwise shaded, to indicate that progress. As a result, a new explicit achieved goal data indicator 819, with a new form and location, along with an abutting pointer 906, are provided, corresponding with the point in time when that amount goal data was to be achieved (i.e., at about the 3^{rd} hour of the time period, or the 3:00 position). Thus, both the deficit indicator, now shown as example deficit indicator 821, and the explicit instruction/verbal phrase 723 (now shown as explicit instruction/verbal phrase 823) and emphatic arc (now shown as decreased demonstrative GUI sub-tool 825) now show a decreased deficit—namely, a deficit of just one (1) cup.

FIG. 9 depicts the same form of tracking indicator 117 as pictured previously, in FIGS. 7 and 8, at an earlier point in time, with the user having achieved all goal data and/or actions required for that point in time. More specifically, in the example provided, the user was required by tracking indicator 117 to consume, and has consumed, two (2) cups of water, and thus has so met the goals prescribed for her or him at that point in time. As a result, pointer 906 has morphed into a form approximating the end 907 of, and merging with hour hand 703 to create, a check mark 909. Emphasizing this achievement, an additional, explicit notification 911 also appears, stating, verbally, that the user is “All Caught Up!”

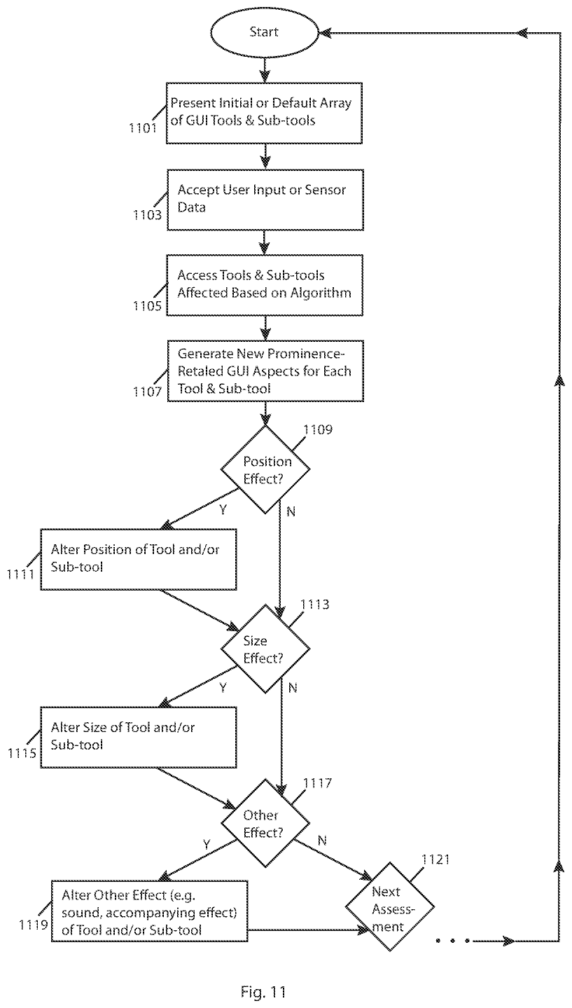

FIG. 10 is a schematic block diagram of some example elements of an example control system 1000, including computer hardware and preferably incorporating a non-transitory machine-readable medium, that may be used to implement various aspects of the present invention, some of which aspects are described in reference to FIGS. 1-9, above, and FIGS. 11-16, below. The generic and other components and aspects described herein are not exhaustive of the many different control systems and variations, including a number of possible hardware aspects and machine-readable media, that might be used, in accordance with embodiments of the invention. Rather, the control system 1000 is described herein to make clear how aspects may be implemented, in some embodiments.

Among other components, the control system 1000 may include an input/output device 1001, a memory device 1003, longer-term, deep data storage media and/or other data storage device 1005, and a processor or processors 1007. The processor(s) 1007 is (are) capable of receiving, interpreting, processing and manipulating signals and executing instructions for further processing and for output, pre-output and/or storage in and outside of the control system 1000. The processor(s) 1007 may be general or multipurpose, single- or multi-threaded, and may have a single core or several processor cores, including microprocessors. Among other things, the processor(s) 1007 is (are) capable of processing signals and instructions for the input/output device 1001, to cause a user interface to be provided or modified for use by a user on hardware, such as, but not limited to, a personal computer monitor or terminal monitor with a mouse and keyboard and presentation and input-facilitating software (as in a GUI), or other suitable GUI presentation system (e.g., on a smartphone touchscreen, and/or peripheral device screen, and/or with other ancillary sensors, cameras, devices, any of which may include user input hardware, as discussed elsewhere in this application with reference to various embodiments).

For example, in some embodiments, camera(s) or other sensor(s) and other user interface aspects may gather input from a user and present user(s) via verbal interactions (e.g., speech recognition and translation), observation techniques and/or with selectable options, such as preconfigured commands or data input tools and sub-tools, to interact with hardware and software of the control system and monitor a user's personal health, environment and data relevant thereto (e.g., food consumption, medication consumption and adherence to health-related personal regimens, and other user behaviors, biomarkers, data and extrapolations from those data, at particular times). For example, in some such embodiments, a user may interact with the control system through any of the actuation and user interface techniques set forth in this application, such as by verbal interaction and/or actuating tools and sub-tools of a GUI (such as any of the GUIs set forth in this application) to run experiments, record data related to her or his personal health, behavior, consumption, biomarkers and environment, causing the control system to record those data and other extrapolations therefrom, or to carry out any other actions set forth in this application for a control system. The processor(s) 1007 is/are capable of processing instructions stored in memory devices 1005 and/or 1003 (or ROM or RAM), and may communicate via system buses 1075. Input/output device 1001 is capable of input/output operations for the control system 1000, and may include and communicate through innumerable possible input and/or output hardware, and innumerable instances thereof, such as a computer mouse(s), or other sensors, actuator(s), communications antenna, keyboard(s), smartphone(s) and/or PDA(s), networked or connected additional computer(s), camera(s) or microphone(s), mixing board(s), reel-to-reel tape recorder(s), external hard disk recorder(s), additional movie and/or sound editing system(s) or gear, speaker(s), external filter(s), amp(s), preamp(s), equalizer(s), filtering device(s), stylus(es), gesture recognition hardware, speech recognition hardware, computer display screen(s), touchscreen(s), sensors overlaid onto touchscreens, or other manually actuable member(s) and sensor(s) related thereto. Such a display device or unit and other input/output devices could implement a program or user interface created by machine-readable means, such as software, permitting the system and user to carry out the user settings and other input discussed in this application. Input/output device 1001, memory device 1003, longer-term, deep data storage media and/or other data storage device 1005, and processor or processors 1007 are connected with and able to send and receive communications, transmissions and instructions via system bus(es) 1075. Deep data storage media and/or other data storage device 1005 is capable of providing mass storage for the system, and may be a computer-readable medium, may be a connected mass storage device (e.g., flash drive or other drive connected to a U.S.B. port or Wi-Fi), may use back-end or cloud storage over a network (e.g., the Internet) as either a memory backup for an internal mass storage device or as a primary memory storage means, and/or may simply be an internal mass storage device, such as a computer hard drive or optical drive.

Generally speaking, the control system 1000 may be implemented as a client/server arrangement, where features of the invention are performed on a remote server, networked to the client and made a client and server by software on both the client computer and server computer.

Control system 1000 is capable of accepting input from any of those devices and/or systems set forth by examples 1009 et seq., including, but not limited to—internet/servers 1009, local machine 1011, cameras, microphones and/or other sensors 1013/1014, Internet of thigs and/or ubiquitous computing device(s) 1015, commercial or business computer system 1017, and/or App-hosting PDA and related data storage device 1019—and modifying stored data within them and within itself, based on any input or output sent through input/output device 1001.

Input and output devices may deliver their input and receive output by any known means, including, but not limited to, any of the hardware and/or software examples shown as internet/servers 1009, local machine 1011, cameras, microphones and/or other sensors 1013/1014, Internet of thigs and/or ubiquitous computing device(s) 1015, commercial or business computer system 1017, and/or App-hosting PDA and related data storage device 1019.

While the illustrated example control system 1000 may be helpful to understand the implementation of aspects of the invention, any suitable form of computer system known in the art may be used—for example, a simpler computer system containing just a processor for executing instructions from a memory or transmission source—in various embodiments of the invention. The aspects or features set forth may be implemented with, and in any combination of, digital electronic circuitry, hardware, software, firmware, modules, languages, approaches or any other computing technology known in the art, any of which may be aided with external data from external hardware and software, optionally, by networked connection, such as by LAN, WAN or the many connections forming the Internet. The system can be embodied in a tangibly-stored computer program, as by a machine-readable medium and propagated signal, for execution by a programmable processor. Any or all of the method steps of the embodiments of the present invention may be performed by such a programmable processor, executing a program of instructions, operating on input and output, and generating output and stored data. A computer program includes instructions for a computer to carry out a particular activity to bring about a particular result, and may be written in any programming language, including compiled and uncompiled and interpreted languages and machine language, and can be deployed in any form, including a complete program, module, component, subroutine, or other suitable routine for a computer program.

FIG. 11 is a process flow diagram, setting forth several example steps 1100 that may be undertaken by a control system (such as the example control system set forth above, in reference to FIG. 10) implementing some aspects of the present invention, according to some embodiments. As discussed above, several devices, systems and methods set forth in the present application relate to GUI tools and sub-tools and other Digital Therapeutics specialized for recording health-related data based on input by users and other sources and administering actions serving as prompts, instructions, or other perceptible interventions impacting the user. In some such aspects, and also as discussed above, some of those tools and sub-tools, and other Digital Therapeutics, may alter their prominence, or otherwise change their presentation, effects and other aspects, based on such data and input, over time. The example steps set forth in reference to this figure are one example embodiment of how a control system running computer software might manage such alterations in prominence and presentation. As will be readily apparent to those of skill in the art, a wide variety of alternative arrangements, steps, number of steps, sequences, and orders of steps also fall within the scope of the invention, and the exact steps, number of steps, sequences, orders of steps set forth herein are but one example, and do not limit the scope of the invention and disclosure.

Beginning with step 1101, the control system first presents an initial or default array of GUI tools and sub-tools, as described immediately above, to a user of the control system. For example, as discussed in greater detail elsewhere in this application, in some embodiments such GUI tools and sub-tools may be specialized for tracking indicators, such as the example tracking indicators shown as 107, which are initially provided in such an initial, default array (such as the array shown as grid 111, in which each of such tracking indicators 107 have the same overall size and shape and a preset starting location, or another default size, shape and location) which may become altered over a time period, as shown, for example, as altered tracking indicators 307.

Proceeding to step 1103, the control system may next enter a mode in which it accepts or reviews health-related data and user activities (e.g., from user input or sensors), and records data related to it, over a time period. In some embodiments, such health-related data, user activities, and algorithms applied thereto may create additional data and actions by the control system, applying Digital Therapeutics to the user. For example, in some embodiments, the control system compares those health-related data and user activities to pre-set goals, also recorded within the control system, as discussed elsewhere in this application. In some such embodiments, the control system creates Digital Therapeutics based on such comparisons. In some such embodiments, such Digital Therapeutics may be altered based on the amount and urgency of a difference between such health-related data, activities and goals, as also discussed in greater detail above. For example, in some embodiments, a goal data point that is associated with a positive health condition or trait compares positively with such health-related data and activities if the health-related data and activities indicate an amount of consumption of a thing, or activity, that meets such a goal data point, as also discussed above. Conversely, in some embodiments, a goal data point that is associated with a positive health condition or trait compares negatively with such health-related data and activities if the health-related data and activities indicate an amount of consumption of a thing, or activity, that fails to meet such a goal data point, or fails to meet such a goal data point by a particular threshold, or, in some embodiments, that greatly exceeds such a goal data point, as also discussed above. In any event, in some embodiments, an algorithm may be applied by the control system, based on such comparisons, which further determines which tools and sub-tools of the GUI should be altered (e.g., differently presented), over time. Such an assessment is carried out by the control system in step 1105. For example, and as also discussed above, such an algorithm may alter the perception of such tools and sub-tools by a user, in some embodiments. In some such embodiments, and also as discussed above, the appearance or effect of such tools and sub-tools may be affected (e.g., by altering size, perceivable location, color, associated sounds, haptic feedback, or other aspects of effects related to the prominence of such tools and sub-tools). The control system my so alter the perception (e.g., prominence) of all such GUI tools and sub-tools by altering the appearance and effects related to those tools and sub-tools in a variety of different possible ways, in subsequent step 1107, based on that assessment.

For example, in some embodiments, as shown in subsequent example step 1109, the control system determines whether the position (or relative position) of any such GUI tool and sub-tool should be altered to alter its perception and/or effect (e.g., to become more or less prominent, e.g., by relocating the tool or sub-tool in a location that is, respectively, more or less central within a display screen controlled by the control system, as discussed in more detail elsewhere in this application). If so, the control system may then proceed to so alter the position, and perception and/or effect, of each such tool or sub-tool, in step 1111. If not, by contrast, in some embodiments, the control system may leave the position of each GUI tool and sub-tool as it was previously, and proceed to example step 1113.

Likewise, in step 1113, the control system next determines whether the size (or relative size) of any such a GUI tool and sub-tool should be altered to alter its perception and/or effect (e.g., to become larger or smaller, to become more or less prominent, respectively, as also discussed elsewhere in this application.) If so, the control system may then proceed to alter the size, and perception and/or effect, of each such tool or sub-tool, in step 1115. If not, by contrast, in some embodiments, the control system may leave the size of each GUI tool and sub-tool as it was previously, and proceed to example step 1117.

As with control system alterations to the location and size of tools and sub-tools, discussed above, the control system may similarly proceed to assess whether any other visible or other aspect or effect of such tools or sub-tools, or other forms of Digital Therapeutics, should be altered, in a series of additional triplets or other sub-sets of steps, such as the example triplet of steps 1117, 1119 and 1121. Thus, in example step 1117, the control system may next determine whether any such additional aspect or effect of any such GUI tool and sub-tool should be altered to alter its perception and/or effect (e.g., to become more or less prominent.) If so, the control system may then proceed to alter that aspect or effect, and perception thereof, of each such tool or sub-tool or other Digital Therapeutics, in step 1119. If not, by contrast, in some embodiments, the control system may leave each GUI tool and sub-tool as it was previously, and proceed to any additional example assessment step, as step 1121, etc.

Following any number of such sub-sets of steps, related to any number of aspects or effects for such tools or sub-tools, or other Digital Therapeutics, the control system may return to the starting position.

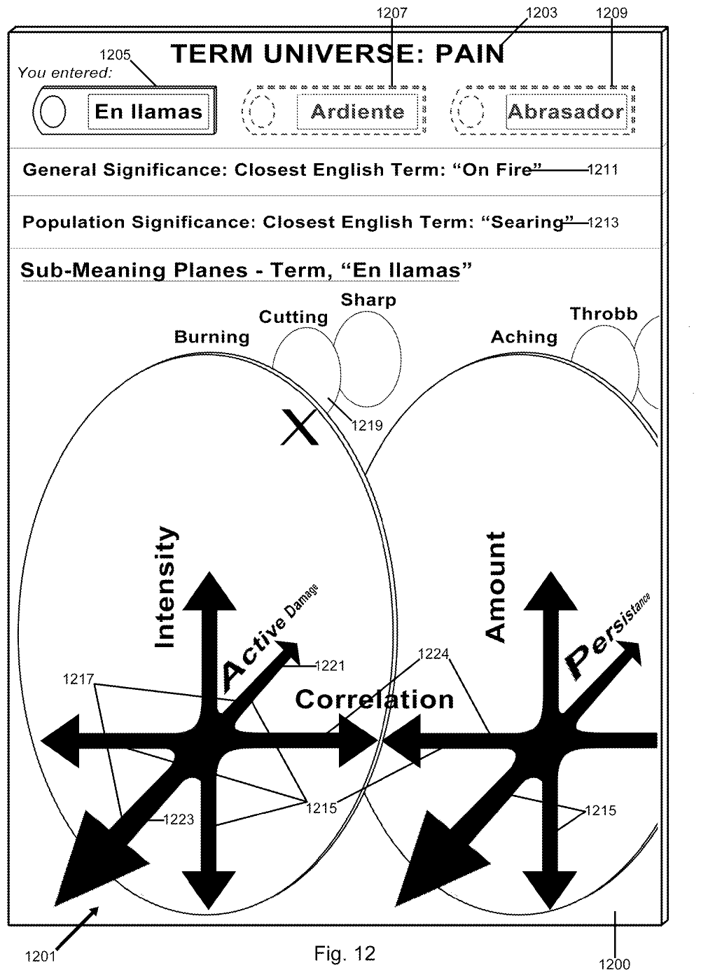

FIG. 12 is a front view of an example GUI 1200 presented by a computer hardware and software control system, implementing some aspects of the present invention related to monitoring and gathering data related to a user (e.g., personal health data and behavior), in accordance with some embodiments. As with other embodiments set forth in this application, GUI 1200 may be presented and implemented through a display device and/or other computer hardware and software used in connection therewith (e.g., on a smartphone or other PDA) in some embodiments. The example GUI 1200 includes a depiction of example aspects of a Significance Map 1201, which is a form of GUI tool configured for managing manual data entries and generating and recording standardized data by such a control system based on a wide variety of linguistic terms entered as input from a plurality of users. As used in this application, a “Significance Map” includes a plurality computer-based logical links between: 1) meanings and sub-meanings of a variety of human language terms and 2) language-neutral codes for new standard conceptual meanings related to a person (or other animal's) health. In some embodiments, as explained in greater detail below, when a user records information by inputting linguistic terms through the control system (e.g., in a GUI allowing for such data input as a basis for generating Digital Therapeutics) such a Significance Map represents the translation of that information into standardized data (a.k.a., a “Translation Vector”). In some such embodiments, such standardized data is then recorded by the control system, and then serves as a basis for algorithms and other software and hardware techniques for delivering Digital Therapeutics, as will be explained in greater detail below.

The example Significance Map depicted in FIG. 12 relates to a general conceptual universe, as shown by universe code 1203—namely, the conceptual universe of “Pain.”While in the English language, the word “pain” may be considered to mean something more broad, and with numerous differing, and potential specified senses set forth within dictionaries, the term “Pain,” as shown in universe code 1203, is instead a code linked or otherwise associated by the control system with a variety of sub-codes, which, themselves, are associated by the control system to any conceptual meanings or sub-meanings relating to negatively perceived sensations or emotional feelings. Although the example universe code 1203, bearing the code “Pain,” and some example sub-codes, conceptual meanings, and sub-meanings relating to negatively perceived sensations and feelings, are provided in and discussed with reference to the present figure, it should be understood that a wide variety of different codes and conceptual areas may, instead, be organized by a control system through any number of similar Significance Maps, related to any such universe codes, each of which Significance Maps and universe codes may be similarly managed by the control system, as set forth further herein.

In the example provided, a user may be entering data relating to the “Pain” code into the control system using GUI 1200 using a term in his or her native language—in the example provided, the Spanish language. In some embodiments, the user may so enter data verbally, by speaking into a microphone—for example, upon a prompt by the control system to enter such terms in connection with creating a record of tracked sensations (among other health-related data recorded and tracked, and basing Digital Therapeutics treatments as set forth in this application). In some embodiments, a user may so enter such terms using a keyboard, mouse and/or touchscreen included within, or in communication with, the control system. In some embodiments, a user may enter such term(s) indirectly, and the term entry in created by the control system, based on other data related to the user's health and/or behavior (e.g., in some embodiments, if a user gasps through her or his teeth creating a hissing sound, after touching a flame or other high-temperature heat source, which are detected by microphones and cameras within the control system, and determined by the control system to be a behavior related to the significance of the term “searing”). In any event, regardless of the method of entry, the user has entered the Spanish term “en llamas,” as shown by example entered term indicator 1205 within GUI 1200, to describe a feeling which she or he is presently experiencing. A wide variety of other terms, and qualifying or localizing terms (locating the source of the pain referred to by the term on the user's body) may also, or alternatively, be used in such data entry by the user, in some embodiments, and the entry of this single term is, of course, merely one example.

Similarly, based on data collected from a wide variety of users of the control system (e.g., through different Software-as-a-Service accounts), many terms may be commonly used by those users to express similar perceived sensations. In some embodiments, the control system associates different terms, to different degrees (e.g., using a correlation algorithm) based on the number of instances of common usage. In some embodiments, such an association and/or algorithm is also based on users manually indicating (e.g., through a GUI aspect) that terms are associated. Terms so associated with such a term that is entered may provide sub-meanings of the term, in some embodiments. Thus, after a population of users has used a variety of pain-related terms over time, a Significance Map (in other words, an outline of meanings and sub-meanings of each term, and correlations and other relations thereof to other terms) is created by the control system—such as the Significance Map 1201, which will be discussed in greater detail below. In some embodiments, the most closely related term(s) (e.g., with most strongly-correlated usage by the users) to each term may be recorded within and added to GUI 1200. For example, in some embodiments, a series of closely related term indicators, such as term indicator 1207 and term indicator 1209, may be created and placed in GUI 1200, presenting those closely related terms to the user—for example, on or about and/or abutting entered term indicator 1205. As different terms entered by users are used more and less often by users of the control system, the exact terms presented in term indicator 1207 and 1209, and the number and rank of such term indicators, may change, becoming more accurate, and reflecting changes in usage by the population of users. In some embodiments, a user may “click on” or otherwise select either of term indicators 1207 and 1209, to enter the terms indicated within them (in this instance, the Spanish words “Ardiente,” indicated in term indicator 1207, and/or “Abrasador,” indicated in term indicator 1209), in addition to, or as an alternative to, selecting the term initially entered by the user (“En llamas”). In this way, the user may select terms that, upon reflection, and in consultation with the entire population of users, best expresses the sensation or emotional feeling she or he is experiencing, and record it with the aid of the control system.

In some such embodiments, a term most closely-related to the entered term may be determined by the control system and provided within GUI 1200. In some such embodiments, a term in another language (other than Spanish, e.g., English) that is most closely related to the entered term may be so determined and provided—for example, as closest term indicator 1211. As mentioned above, in some embodiments, such terms, the relation of terms may be based on correlated use between the entered term and its most closely related term within a population, as reflected by alternative closest term indicator 1213. However, alternatively, or in addition, and as set forth in the instance provided as closest term indicator 1211, such a closest term indicator may be based on accepted meanings as set forth by professional linguists (e.g., the authors of dual language or other dictionaries and/or other secondary sources of the significance of terms and words) and the correlation of term and word significance of different terms set forth therein. In some embodiments, an administrative user or other secondary user may presented with, and evaluate the significance of, the term entry by the user in one language, by being presented with a closest term indicator, such as the example provided as closest term indicator 1211, or an alternative closest term indicator, such as the example provided as closest term indicator 1213. In some embodiments, the control system may record both the initially entered term, and at least one of closest term indicators 1211 and 1213. In some embodiments, the control system may record both the initially entered term, and each of closest term indicators 1211 and 1213. In some embodiments, the control system may record the entry of such terms and associate a time of day, or other time period, with such an entry or pain sensation, in a database encoded with an account assigned to the user. In some embodiments, secondary users may review such recorded data and metadata, and such user accounts, if they are authorized to view data relating to the user.

In some embodiments, such different term indicators may indicate different meanings. For example, as pictured, closest term indicator 1211 indicates that the closest English term to the entered Spanish term “En llamas” is “On Fire,” according to such secondary sources, but closest term indicator 1213 indicates that the closest English term is actually “Searing,” according to correlation of use by the population of users.

In some embodiments, as with the universe code 1203, any term entered by the user to signify an experience of Pain (by sensation or emotional feeling), as discussed in the example of “En llamas,” above, may be converted to a code, and a new, standardized significance related to that code. In other words, in some embodiments, rather than (or, in some embodiments, in addition to) recording the entry of the term “En llamas” merely as a term used in the Spanish language, the control system may enter “En llamas” as at least one code for a new, different, standard meaning managed by the control system. As mentioned above, such standardized meanings, and sub-meanings thereof, may be each so individually coded and correlated with one another, with their interrelations and degree of correlation recorded as a Significance Map, in some embodiments, such as example Significance Map 1201.

For example, in some embodiments, a sub-meaning of the term “En llamas” is the concept that a burning sensation is sharp, and so sharp as to even be cutting, as experienced by the user. Because flames tend to concentrate their energy in narrow areas as fuel is burned, this relationship is literally experienced when a person is experiencing fire (e.g., accidentally licked by the tip of a fireplace flame) and, thus, such a localized, cutting sensation and association for the term “En llamas” may be commonly observed in a population. In some embodiments, such a sub-meaning may be assigned both to the code “En llamas” and to a sub-meaning, which may be coded as example sub-meaning codes “En llamas/Cutting” and/or “En llamas/Sharp.” In this way, if other users enter other terms, which also have such a standardized sub-meaning associated with it, the same code may be assigned to such data entry. As an alternative to such coding, or in addition to it, in some embodiments, a visual construct of such coding of such relationships may be presented to a user—for example, as a graph incorporating “lines” or “planes” of meaning, as illustrated by example lines of meaning 1215. In some embodiments, such lines or planes of meaning are restricted to a single sub-meaning, which may be included within in any number of data entries by users (e.g., by different terms whose significance each include that sub-meanings.) In this way, a single term or code may be mapped, relative to others, which may share that sub-meaning. For example, as shown in example Significance Map 1201, the term “en llamas” may share a sub-meaning, and illustrated line of meaning, that there is current, active damage to the user being perceived, which line of meaning is illustrated as example line of meaning 1217. Similarly, a Significance Map for another term entered by users, namely “Cutting,” may be included within that line, but at a different location within the Significance Map, as shown by example neighboring Significance Map 1219, shown in a minimized format, in the direction indicated (into the page, or “negative z” axis).

In some embodiments, a user may “navigate” between terms and codes sharing sub-meanings by “clicking on” one or more corresponding GUI arrow sub-tools, which may be provided in multiple directions along such a line of meaning. For example, line of meaning 1217 is shown as including two such sub-tools—arrow sub-tool 1221, for navigation in one direction, and arrow sub-tool 1223, for navigation in a direction opposite to that one direction. In some embodiments, a line of sub-meaning may include a continuum of changing characteristic(s) of the sub-meaning. For example, as a user progresses in the direction of arrow sub-tool 1221, the characteristic of a sharper active damage increases, such that, upon further navigation in that direction, the control system may present a more distant, albeit related, significant map 1225, for the term “Sharp.” In some embodiments, a combination of one or more lines of sub-meaning significance may be referred to as a “plane” of sub-meaning, as illustrated by GUI planes 1227, which may be comprised within a Significance Map. In some embodiments, Significance Maps are individually coded, recorded and modified over time, based on user data (such as the changing correlated sub-meanings of related, as discussed above).

In some embodiments, as with the lines of meaning, and GUI sub-tools dedicated thereto discussed above, Significance Maps may be closely related to one another within planes of meaning. For example, in some embodiments, users within the population of users managed by the control system may access, record or otherwise manage data encoded with a Significance Map in combination with access, record or otherwise manage data encoded with another significance map. In this sense, different Significance Maps, as with different terms, may be correlated with one another. In some embodiments, this correlation may be expressed as a line of meaning based on that correlation, such as correlation line of meaning 1229.

In some embodiments, the user entering the term to record health-related data, or a secondary (e.g., administrative or authorized health professional) user may select or deselect such relationship, removing or recording their significance, and associating or disassociating them with the term entry by the user.

The totality of all Significance Maps managed by the control system, with all relationships between one another recorded, navigable, selectable and de-selectable, assisting in recording any known sensations or emotional feelings of the population of users, as set forth above, is referred to herein as a “Total Significance Map.”

FIG. 13 is a perspective view of an example environment 1300 in the process of being monitored by one or more example imaging sensor(s) 1301, which may be controlled by a control system including computer hardware and software (such as any of the control systems set forth in this application), in accordance with some embodiments. In some embodiments, such an imaging sensor(s) 1301 may be any suitable form of sensor for capturing an image and/or detecting and recording image data from an environment. For example, in some such embodiments, imaging sensor(s) 1301 include a wide-angle imaging sensor, meaning that it is configured to take in a substantial proportion of the environment that it is placed in, on or about.

In some embodiments, such a wide-angle imaging sensor is capable of capturing an image of at least a 90-degree view of such an environment. In some embodiments, such a wide-angle imaging sensor is capable of capturing an image of at least a 120-degree view of such an environment. In still other embodiments, such a wide-angle imaging sensor is capable of capturing an image of at least a 180-degree view of such an environment. In still other embodiments, such a wide-angle imaging sensor is capable of capturing an image of at least a 270-degree view of such an environment. In still other embodiments, such a wide-angle imaging sensor is capable of capturing an image of at least a 360-degree view of such an environment.

In some embodiments, imaging sensor 1301 includes at least one imaging, range-finding or other device for detecting the presence and/or nature of objects and/or activity within an environment. In some embodiments, imaging sensor 1301 includes a camera. In some embodiments, imaging sensor 1301 includes an infrared sensor. In some embodiments, imaging sensor 1301 includes a rangefinder. In some embodiments, imaging sensor 1301 includes a L.I.D.A.R. device. In some embodiments, imaging sensor 1301 includes a R.A.D.A.R. device. In some embodiments, imaging sensor 1301 includes a thermometer. In some embodiments, imaging sensor 1301 includes a lens. In some embodiments, imaging sensor 1301 and/or the control system managing imaging sensor 1301 performs object recognition methods on image information it captures. As will be explained in greater detail below, in some such embodiments, such a control system maintains a library of data associated with particular objects or classes of objects, and compares image and other data it captures in real time with such data related to particular objects or classes of objects, thereby matching objects detected within an environment to particular objects or object types. As will also be discussed in greater detail below, in some embodiments, the control system analyzes image and other data captured by imaging sensor 1301 in real time for changes in size, contents, or other consumption and activity-related conditions, and then creates a record of such consumption and activity by a user. In some embodiments, the control system analyzes image and other data captured by imaging sensor 1301 in real time for the presence and activity of a user (e.g., food consumption or exercise), using similar comparisons to pre-recorded image and other data related to the user (e.g., facial recognition techniques). In some embodiments, the control system monitors a user's biometrics, biomarkers or other indicators of the user's current health-related data, status or other condition. In some embodiments, the control system and/or imaging sensor 1301 captures imaging data of substantially all physical activity of any matter viewable within an environment. In some such embodiments, imaging sensor 1301 includes matter-penetrating imaging techniques (e.g., X-ray or ultrasonic imaging devices). In some embodiments, imaging sensor 1301 includes a combination of two or more devices listed above.

In some such embodiments, and also as discussed in greater detail below, the control system may search and determine such matter, objects, conditions thereof and activities by users at a later time (e.g., by comparison to later-acquired object-, user- and activity-related data). In some embodiments, the control system may identify potential causes, or complexes thereof, (a.k.a., hypotheses) from correlations of objects and activities detected in an earlier observed time, to conditions of a user, detected at a later-observed time. In some such embodiments, a repeated or otherwise strong correlation of such potential causes with such conditions of a user may give rise to higher priority hypothesis, which may be presented to a user and/or administrative user (e.g., a physician or other health care personnel).

As will also be discussed in greater detail below, in some embodiments, the control system manages a plurality of other such imaging sensors, similarly monitoring other environments, and objects and users therein. In some such embodiments, data related to environments, objects and users that are grouped together in some way may be linked and analyzed together in a single study (e.g., a retroactive experiment). In some embodiments, hypotheses developed, at least in part, from detecting one user's condition(s) and/or environment(s) may be presented to another user, based users' conditions and/or potential causes.

In any event, in the example pictured, environment 1300 includes an example food container—namely, box 1305 of granular food particles 1307, placed on a kitchen counter 1309. By observing box 1305 from a variety of angles over time, and passing related imaging data over time, with time stamps, to the control system, the control system can assess the amount of food present, the type of food present (e.g., by optical character recognition (“OCR”) of text on the box label hardware device 1311, and/or by comparison to image data related to such food particles or types thereof stored in an object library) and the consumption of that food by a user (e.g., by user activity recognition). Such consumption and user activity recognition may be aided by control system recognition (e.g., via machine vision and/or additional artificial intelligence techniques) of ancillary objects (e.g., nearby consumption-indicating objects, such as example spoon 1313 and example bowl 1314). By observing the emptying of such consumption-indicating objects, in some embodiments, the control system may also determine a more precise time and rate of consumption of food particles 1307 by a user (not pictured).

In some embodiments, box label hardware device 1311 is a label comprising scannable hardware and information transmission technology. In some embodiments, such information transmission technology includes a code, such as a unique optical pattern 1312, disposed on its outer surface. In some embodiments, box label hardware device 1311 also includes a food scanning sub-device, disposed on an inside surface of the box label hardware device 1311. In some such embodiments, such a food scanning sub-device is integral with, or disposed on, an interior surface of a food container, such as box 1305. In some embodiments, unique optical patter 1312 includes a control system including a dynamic display technology (e.g., an e-ink display) that changes to code for and/or reflect information regarding the contents of such a food container. In some such embodiments, such information regarding the contents includes a fill level of the food container. In any event, by scanning the unique optical pattern 1312, sensors 1301, and a control system comprising or comprised in them can readily determine the amount and type of food present within the food container, in some embodiments, at particular times. By assessing changes in such a fill level and/or contents, and the identity of a user present within environment 1300 at those times, a food consumption rate, relative to the food present within the food container, can be determined. Base on such consumption rates, health-related data can then be recorded, and serve as the basis for Digital Therapeutics techniques set forth in this application. In some embodiments (as pictured) box label hardware device 1311 is disposed on at least one corner or other vertex of a food container (such as the side box corner 1315). In some such embodiments, the unique optical pattern is repeated on surfaces substantially disposed over multiple sides of box 1305. In some embodiments, such a vertical pattern is not repeated on multiple sides of box 1305, but is presented in a format visible from multiple sides of box 1305. In any event, by presenting a unique optical pattern disposed from different sides of box 1305, there is a greater likelihood that one or more of imaging sensors 1301 will be able to sense, and obtain a reading of that unique optical pattern, which can then form the basis of Digital Therapeutics measures, as set forth in this application.

Of course, the example of a kitchen or other food consumption environment (environment 1300) and food-related activity is just one of virtually unlimited possible environments and activities that may be similarly tracked in accordance with many alternate embodiments set forth in the present application. For example, in some embodiments, the environment observed may be a gym or other personal exercise environment, and the activity observed may relate to physical exercise, with observations of objects, materials and other indicators of such physical exercise. In other embodiments, the environment observed may relate to any particular human activity, objects or materials that is relevant to the health of a user.

Imaging sensors 1301 may take on a wide variety of form factors, to enhance their operation, in addition to the form factors pictured. However, in some embodiments, multiple corner-filling formats are presented, some of which embodiments may include multiple (or all) distal ends or edges, such as the example edges 1317, which taper seamlessly, creating a flush surface with, surfaces, such as example surfaces 1319, of the walls 1321, ceiling 1323, or other surfaces of environment 1301.

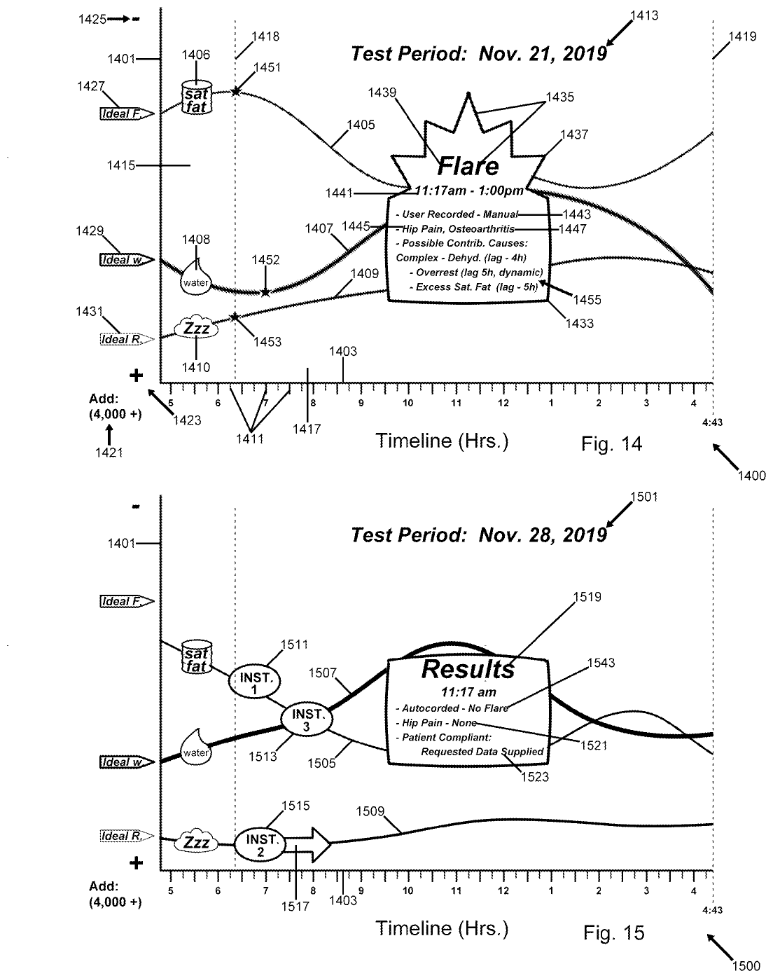

FIG. 14 is a front view of an example user interface 1400 incorporating graphical aspects, which display personal health-related data over time for a user of hardware and software aspects set forth in the present application. Among other things, user interface 1400 may include a plurality of graph axes, such as example vertical axis 1401 and example horizontal axis 1403, each of which abutting and providing context to example line graph GUI tools 1405, 1407 and 1409. In some embodiments, each of example line graph GUI tools 1405, 1407 and 1409 plot data related to an amount or degree of some consumption, activity or other health-related aspect of the user and/or her or his life or environment, over time. Thus, horizontal axis 1403 plots the progress of time, from left to right, by regular intervals indicated by periodic ticks (such as the example ticks 1411) with line graph markings at each horizontal position indicated by those ticks indicating an amount or degree of some health-related aspect of the user and/or her or his life or environment by vertical position. Ipso facto, the vertical axis 1401 plots the amount or degree of each health-related aspect of the user and/or her or his life or environment at particular times by line graph markings at vertical positions.

In particular, line graph GUI tool 1405 plots data representing a rate, or average rate at a particular time, of saturated fat consumed by the user during a data gathering period covered by user interface 1400. In some embodiments, a time period indicator 1413 is included, indicating to the user that user interface 1400 relates to such a data gathering period. In some embodiments, such an average rate is a trailing average rate (e.g., an average hourly rate of consumption for the previous two hours at the time, a “trailing two hours” indicator). To aid a user in understanding that line graph GUI tool 1405 relates to such saturated fat consumption by the user, a line tool label 1406 may be provided, in some embodiments. In some embodiments, such a line tool label 1406 is presented in a specially reserved guidance area 1415, adjacent to a data examination area 1417. Data examination area 1417 presents indicators, data and other GUI Tools and sub-aspects that are active—meaning that an analysis has been or is currently being run, at least some results of which, and/or GUI tools in connection to which, are being presented within, data examination area 1417. Preferably, in some embodiments, data examination area 1417, by contrast, does not contain at least the main or direct results of such an analysis. Similarly, to aid a user in understanding that line graph tool 1407 and line graph tool 1409 relate to water consumption by the user and sleep or other rest by the user, respectively, line tool label 1408 and line tool label 1410 may be provided, respectively. Also similarly, line tool label 1408 and/or line tool label 1410 may each be presented within examination area 1417, in some embodiments. As alluded to above, line graph GUI tool 1407 plots data representing a rate, or average rate at a particular time, of water ingested by that user, or otherwise taken into her or his body, during the data gathering period covered by user interface 1400. In some embodiments, such an average rate is a trailing average rate (e.g., an average hourly rate of consumption for the previous two hours at the time, a “trailing two hours” indicator). Also as alluded to above, line graph GUI tool 1409 plots data representing a rate, or average rate at a particular time, of rest undertaken by that user during the data gathering period covered by user interface 1400. In some embodiments, such an average rate is a trailing average rate (e.g., an average hourly rate of consumption for the previous two hours at the time, a “trailing two hours” indicator).

In some embodiments, a starting boundary 1418, extending vertically at the horizontal position along axis 1403 corresponding with the point in time at which data relevant to such an analysis, marks the boundary between specially reserved guidance area 1415 and data examination area 1417. Similarly, in some embodiments, the current time and/or a final time in which data relevant to the analysis is presented, may be indicated by another, ending boundary line 1419.

Although the example of three (3) line graph GUI tools is provided in the present figure, it should be understood that many more, or fewer, such line graph tools may be provided, plotting data relative to virtually any possible health-related aspect of that user and/or her or his life or environment. In some embodiments, that user, or another, authorized user, may add additional line graph GUI tools, tracking any such possible health-related aspect (or group of aspects, in some embodiments in which indexes of groups or types of such data, are created by an algorithm applied by the control system), or remove them. For example, in some embodiments, a user may click on or otherwise activate a line graph addition sub-tool 1421, which may then lead to a GUI tool presenting a list of such selectable aspects, and cause GUI line tools related to any one of them to be presented within GUI 1400.

As mentioned above, some data presented with the aid of a graphical axis may be presented in regular, metered units, in some embodiments. In some such embodiments, some such data may be indicated by units marked on such an axis, such as on axis 1403, by the example ticks 1411 (indicating units of time—namely, hours, and regular sub-divisions thereof, with counting (arithmetic) indicators). However, in some embodiments, an axis may not indicate data by such marking, or as arithmetic, regular expression of such data. For example, where, as in the embodiment pictured, different forms of data, with different units, are commonly expressed in relation to such a graph axis, such ticks or other regular indicators may be omitted. In such embodiments, each line graph GUI tool may be expressed according to its own method, via a separate scaling function, to present substantially all relevant data for such a line graph tool, while still accurately indicating trends or other directional changes in such data over the data gathering period. In some such embodiments, the expression of such each line graph GUI tool may be logarithmically, or otherwise adjusted by an algorithm to create such a common, directional presentation of each respective represented data set.

In some embodiments, some line graph GUI tools may be initiated on a lower or higher point along vertical axis 1401, depending on whether the data relates to an aspect considered generally good or bad, respectively, for the user's health when achieved by the user. For example, as pictured, line graph GUI tool 1405 is initiated on the upper side of vertical axis 1401, because line graph GUI tool 1405 relates to saturated fat consumption by that user, which is generally considered to negatively correlate with users' health, and increasing amounts are shown as a negative (higher) corresponding vertical levels (i.e., multiplied with an arithmetic sign to cause such an expression) within data examination area 1417. To indicate the nature of such placements and/or sign assignments, a positive aspect indicator 1423 and a negative aspect indicator 1425 may be placed at lower and higher positions, respectively, for such aspects. In some embodiments, each line graph GUI tool may have an inverse version (not pictured) relating to an opposite aspect (e.g., an excess of or a lack) of the aspect indicated by line graph GUI tool. For example, in some embodiments, water consumption, which is generally considered a positive aspect for a user's health and, thus, expressed by line graph GUI tool 1407, initiated on the lower end of vertical axis 1401, may be expressed as a lack of water consumption, or an overconsumption of water, which may be initiated instead on the negative end of vertical axis 1401, if selected for presentation within GUI 1400.

In some embodiments, ideal rates of occurrence of aspects tracked by each line graph GUI tool may be presented within GUI 1400. For example, with respect to line graph GUI tool 1405, an ideal rate indicator 1427 may be included, indicating the ideal rate of consumption of saturated fat for the user. Thus, by referring to the vertical level of ideal rate indicator 1427 along vertical axis 1401, a user can rapidly determine whether her or his current rate of consumption of saturated fat is higher or lower than the ideal rate, and adjust her or his consumption accordingly. Similarly, ideal rate indicator 1429 and ideal rate indicator 1431 may also be included, in some embodiments, similarly rapidly indicating to the user if her or his consumption of water and rest, respectively, match an ideal rate indicated by their vertical positions along vertical axis 1401.

In some embodiments, such ideal rates may change over time (a.k.a., “floating indicators”), based on an ongoing condition assessed or sensed for a user and/or based on data derived from in-body experiments and their results, for the user and/or an entire cohort of users (e.g., designated by an administrative user conducting a mock experiment. Such aspects will be discussed in greater detail below. Thus, in embodiments discussed in greater detail below, each of floating ideal rate indicators 1427, 1429 and 1431 are each altered slightly, indicating a different vertical position and amount than previously, at a later time period.

As discussed above, in some embodiments, GUIs presented to a user contain Digital Therapeutics, which may include information and guidance to the user regarding the presence or absence of adverse and positive health conditions of that user. As one example, in the embodiment pictured in the present figure, information and guidance relating to ideal levels of activity and consumption are provided by ideal rate indicators 1427, 1429 and 1431. As another example, adverse health events may be indicated to the user, in some embodiments. For example, in some embodiments, in addition to tracking consumption and activities of the user, markers of adverse events may be sensed (e.g., by cameras or other imaging devices within the control system monitoring user behavior and/or visible markers of such adverse events.) For example, in some embodiments, the control system implements adverse event recognition algorithms, and compares currently sensed data with stored data associated with adverse events (either for that particular user and/or for other users of the control system within a particular cohort.)

Thus, and, again, as one example, an event indicator, such as example adverse event indicator 1433, is included within GUI 1400, in some embodiments. Among other possible sub-tools, adverse event indicator 1433 may include the nature of the adverse event, via one or more adverse event type indicators 1435. Such adverse event type indicators may be symbolic, as shown by example pointed explosion symbolic indicator 1437, in some embodiments. In some embodiments, such adverse event type indicators may be in a written language form, such as flare sub-indicator label 1439.

In some embodiments, such an adverse event indicator may be placed directly over a region of data examination area 1417 at horizontal positions coinciding with the time period during which the adverse event is detected (or entered or otherwise indicated by the user). In some embodiments, that time period is further illustrated by an adverse event time period indicator 1441. In some embodiments, if a user manually enters data indicating that the underlying adverse event has occurred, that manual type of data entry is indicated by a data entry type indicator 1443. In some embodiments, by contrast, if the control system senses or otherwise determines data indicating that the underlying adverse event has occurred, that automatic type of sensing and recordation is indicated by an alternate data entry type indicator, similar in nature to the alternate data entry type indicator 1543, discussed in reference to FIG. 15. In some embodiments, adverse data indicator 1445 is included within adverse event indicator 1433, indicating a type of sub-condition and/or data which is a basis for the control system's determination that an adverse event has occurred. Thus, adverse data indicator 1445 indicates that the user has recorded hip pain in one or more of her or his hips, which has been related to an auto-immune flare-up of her or his immune system (e.g., arthritis) within the user's body. In some embodiments, a condition type indicator 1447 may be included, indicating an overall disease or other ongoing health condition of the user, tied to the adverse event and/or adverse data. Thus, to extend the example, condition type indicator 1447 indicates that a user is an “osteoporosis” sufferer, which contributed to the occurrence of the user's hip pain and/or immune system flare-up.

As also mentioned elsewhere in this application, in some embodiments, correlations between adverse health events and aspects of a user's health may be determined by a control system incorporating a Digital Therapeutics GUI, such as GUI 1400, in some embodiments. Furthermore, assessments of potential causation between such events and aspects may also be determined by the control system, in some embodiments. In some embodiments, such a causation of an adverse event by such an aspect may be a hypothesis, based on known causal relationships supported in 1) peer-reviewed medical literature, establishing scientific facts; 2) data gathered and causal relationships determined or suspected between similar adverse events and similar aspects determined for other users of the control system; and 3) data gathered and causal relationships determined or suspected between similar adverse events and similar aspects for that user. Based on any or all of the above three sources, hypotheses may be generated by one or more hypothesis-generation algorithms, and suspected causes, prior to an adverse event, are identified and labeled, in some embodiments. For example, the control system has assigned three suspected cause indicators within GUI 1400—namely, suspected cause indicator 1451, suspected cause indicator 1452 and suspected cause indicator 1453, identifying suspected causes related to saturated fat overconsumption, water underconsumption and too much rest/inactivity for the user, respectively. Thus, the control system has determined, based on any and/or all sources of data, and by applying hypothesis-generation algorithms, that such suspected causes have occurred. In some embodiments, the control system also determines a particular time at which those causes occurred, or reached a likely critical level (triggering the adverse event) and creates indicators of those times. For example, in some embodiments, each of the suspected cause indicators 1451, 1452 and 1453 are placed along the line graph GUI tool plotting data for the aspect to which it relates—1405, 1407 and 1409, respectively. And, to continue the example, each of the suspected cause indicators 1451, 1452 and 1453 identify data and the time at which a trigger amount of consumption or activity or another aspect occurred which triggered, or first triggered, the adverse event indicated by adverse event indicator 1433. In some embodiments, the timing of such suspected causes may be amplified by additional GUI sub-tools, such as example lag-indicating sub-tools 1455. In some embodiments, lag-indicating sub-tools 1455 indicate the amount of time since the time indicated by each suspected cause indicator, before the time that the adverse event occurred. Thus, the adverse event often may not follow a cause, or complex of contributing causes, immediately, and the lag-indicating sub-tools aid the user in elucidating a number of complex, contributing potential causes. In some embodiments, such causes are not necessarily assigned a particular point in time, and/or no particular point in time for a triggering amount can be identified by the control system. In such cases, a larger period of time in the past may be indicated (e.g., by a colored, highlighted or shaded portion of one or more line graph GUI tools). In some embodiments, such points in time and/or larger time periods are co-dependent with other suspected causes, as determined by the control system. In other words, in the absence of other suspected contributing causes of an adverse event, a suspected cause might have a different point in time, time period, lag, or may not even be determined to exist, relative to the adverse event, depending on the aspects tracked by the control system and/or within GUI 1400. In some embodiments, the control system automatically selects aspects to be tracked by such line graph GUI tools, based on a determination that the aspects they represent likely contributed to a detected adverse event. In some embodiments, the user or an administrative user (e.g., a health coach) may select the aspects to be tracked and analyzed as a suspected cause, forcing the control system to focus only on a selection of suspected causes, and generating each of the GUI tools and sub-tools discussed above.

As discussed above, such potential causes and relationships form the basis of further adjustments in user behavior, using Digital Therapeutics in accordance with the present application, in some embodiments. In some such embodiments, in-body experiments are carried out using such Digital Therapeutics, based on suspected causes, such as those discussed above. Because, in addition to the user of GUI 1400, the control system and Digital Therapeutics techniques set forth in this application may be carried out on a plurality of such users, each of which have unique user profiles and health-related data securely stored, separately, by the control system, a single analysis of multiple users' data may be carried out by the control system, and administrative users of the control system, in some embodiments. In some embodiments, mock experiments and/or retroactive experiments, also may be carried out. Examples of each of these techniques will be discussed in greater detail below.

FIG. 15 is a front view of the same example user interface, now shown in an alternate format 1500, at a later time, with altered tools and sub-tools, and some additional sub-tools, in accordance with some embodiments. Based on the adverse event determined to have occurred in the period of time covered in FIG. 14, the control system has determined to test a similar, or related, albeit different time period. In some embodiments, that new time period is now indicated by a new time period indicator 1501, and is the same day of the week as that covered in reference to FIG. 14, but one week later. Based on the suspected causes previously identified by the control system, with suspected cause indicators 1451, 1452 and 1453, the control system may now seek to reduce the probability of a similar adverse event as discussed above from happening again. In some embodiments, the control system does so by providing guidance, in the form of Digital Therapeutics, which cause the user to prevent the repetition of levels, rates and/or amounts of aspects tracked by the line graph GUI tools, or complexes thereof (or, in some embodiments, patterns thereof and/or correlated or otherwise similar patterns thereof) that contributed to an adverse event in the past. As discussed above, such aspects may be any health- or fitness-related variable, including, but not limited to, food, beverage and other matter consumption, biomarkers (including, but not limited to vital signs and behavioral biomarkers), physical and mental conditions, environmental stimuli, and activity levels (such as, but not limited to, exercise).

In some embodiments, however, the control system may instigate the creation of user-health related data (and aspects such as those discussed immediately above causing that data) for conducting an in-body experiment. In some such embodiments, the control system does not necessarily base Digital Therapeutics on preventing such a repetition of levels, rates and/or amounts of aspects. Instead, or (in some embodiments) in addition, the control system instigates the entry or collection of data related to user aspect(s) it manages by selecting a set of levels to be tested (thus creating an “in-body experiment”). In some embodiments, such selecting is random. In some embodiments, such selecting is partially random. In some embodiments, such selecting is pseudo-random. In some embodiments, such selecting is based on health- and/or fitness-related data of other users of the control system, and correlations of those data with positive and negative outcome data.

In any event, in some embodiments, the control system causes the user to alter her or his health-related aspects, and the entry of particular health- and/or fitness-related data, by providing Digital Therapeutics (or digital test instigations) using GUI sub-tools, termed “instigators,” in some embodiments. For example, in some embodiments, an instigator, as recorded within GUI 1500 as instigation marker 1511, may have been created, relative to the saturated fat line graph GUI tool, now shown in new line graph GUI form 1505, which now reflects the effect of that instigation. In some embodiments, instigations are made in the new time period at a time based on the time that possible causes of an adverse event (or other health-related event related to the Digital Therapeutics presently being carried out) occurred in the past. For example, in some embodiments, a probable time for such a health-related event to occur in the new time period is determined and, based on a lag time (as discussed in greater detail above) for possible causes of such events, an instigation is created in the new time period, directing the user to alter one or more aspects of such a possible cause. More specifically, an instigation GUI tool may have instructed the user, on or about 6 hours and 30 minutes into the new time period, to decrease her or his consumption of saturated fat (e.g., by an indicator presented within such an instigator stating “stop eating the doughnuts,” or whatever other form of food(s) high in saturated fat were then being consumed by the user). As a result of that instigator, the user then decreased her or his consumption of those food(s), leading to a lowering of the rate at which her or his body was consuming and digesting saturated fats, as shown by the line graph GUI form 1505. Similarly, an instigator causing an increase in the user's consumption of water may have been effectuated at a slightly later time (in some embodiments, to counteract a suspected cause later in time in a previous time period), as recorded within GUI 1500 as instigation marker 1513, leading the user to increase her or his water consumption, as shown by an upward-trending new line graph GUI form 1507 of line graph GUI tool 1407.

As another example of an instigation, another form of instigator may have been created relative to the user's levels of rest, as tracked with line graph GUI tool 1409, now shown as new line graph GUI form 1509. In some such embodiments, such an instigator may persist over a period of time, taking on an extended form that continually coaches the user to maintain a target level of such an aspect (e.g., via biofeedback delivered through GUI 1500). The occurrence of such an instigation may be recorded as alternate instigation marker 1515, which may include the duration and direction of such a target level, via a directional sub-marker 1517. In some embodiments, directional sub-marker 1517 may be in the form of an arrow (as pictured) indicating the target level of the health-related aspect for the user for that duration.

Due to the influence of the instigations discussed above, in the new time period indicated by new time period indicator 1501, in some embodiments, the adverse event (or other health-related event) that had occurred in the previous time period may not have recurred in the new time period. As a result, rather than the adverse event indicator 1433, a new results indicator 1533 is presented, in some embodiments, in the same position (or same relative position, based on some common features of GUI 1400 and 1500) within GUI 1500. Thus, rather than indicating the nature of an adverse event, via one or more adverse event type indicators, new results indicator 1533 instead may comprise sub-tools indicating that it is results-oriented—such as results indicator 1519. Other sub-tools of new results indicator 1533 may provide details and other guidance to a subject user or other, administrative user, in some embodiments. For example, in some embodiments, data or health-related activities confirming that the health-related event was avoided in the correlated, new time period, may be included, as with the example asymptomatic indicator 1521, shown within results indicator 1519. In some embodiments, results indicator 1519 may include an indicator (such as a report) of the degree of compliance with Digital Therapeutics guidance by the subject user, as shown by example compliance indicator 1523.

As mentioned above, in some embodiments, instigations are used for different, or additional techniques—other than avoiding the occurrence of health-related events. For example, in some embodiments, the control system may vary instigations (e.g., randomly), and test results for the objective of gaining more general knowledge of health-related aspects, and their interrelations with other factors. For example, in some embodiments, in-body experiments may be carried out. Preferably, in some embodiments, a single variable may be altered (in the form of a single health aspect, or sub-aspect) by such instigations, over different time periods, and potentially caused conditions or events may be later assessed by the control system. Such testing of the results of instigations may be termed “in-body experiments” within the lexicography of this application.

In some embodiments, larger experiments, involving a cohort of a plurality of users of the Digital Therapeutics control system, may be conducted by creating such instigations for multiple users, and assessing commonly occurring, potentially-caused health-related events.

In some embodiments, mock experiments may be conducted, by assessing such possible causes from one or more users (or one or more time periods) and so instigating data for other user(s) (or other time periods).

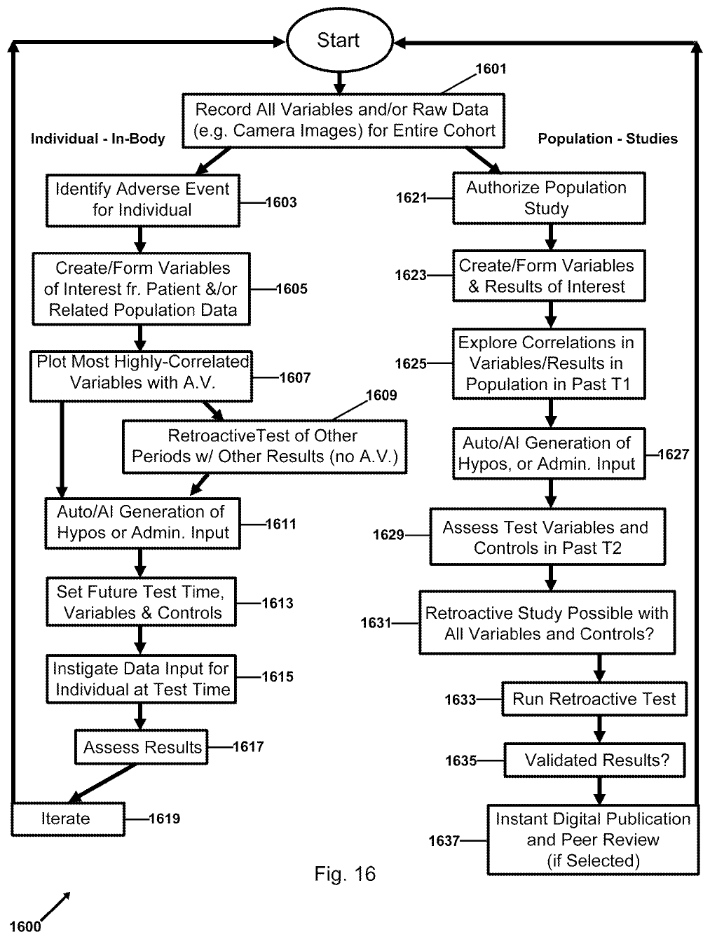

FIG. 16 is a process flow diagram, setting forth several example steps 1600 that may be undertaken by a control system (such as the example control system set forth above, in reference to FIG. 10) implementing some aspects of the present invention, according to some embodiments. As discussed above, several devices, systems and methods set forth in the present application relate to GUI tools and sub-tools specialized for recording health-related data, and for carrying out Digital Therapeutics using such a control system. Also as discussed above, some such embodiments involve instigating the creation of such data, based on a wide variety of factors and for a wide variety of purposes. For example, and as discussed in this application, such instigations may support interventions to alter aspects of possible causes of health events, and other Digital Therapeutics and experiments applied to a single user's body and personal health (a.k.a., “in-body”). As another example, in some embodiments, and also as discussed in this application, such instigations may support more general studies, such as larger experiments involving an entire cohort of users of the control system, and mock experiments. The example steps set forth in reference to this figure are one example embodiment of how a control system running computer software might manage such instigations of data using Digital Therapeutics techniques. As will be readily apparent to those of skill in the art, a wide variety of alternative arrangements, steps, number of steps, sequences, and orders of steps also fall within the scope of the invention, and the exact steps, number of steps, sequences, orders of steps set forth herein are but one example, and do not limit the scope of the invention and disclosure.

As discussed in more detail above, and by virtue of many possible embodiments, such a control system may record a wide variety of health-related data, both from individual users, and from a larger population of multiple users, as set forth in an initial step 1601. In some embodiments, and also as discussed elsewhere in this application, raw data of a complex type may be recorded, such as raw image or video data, recorded by a camera comprised with the control system. In some such embodiments, the control system may analyze such complex data, and extrapolate additional data sets (e.g., at the request of a scientist setting forth data points of interest in a population-wide study, as discussed in greater detail elsewhere in this application.

In any event, in some embodiments, the control system next may enter any of multiple sets of steps related to instigating health-related data for different purposes. For example, in some embodiments, the control system may initiate and complete steps 1603 through 1619, if it receives a request from a user, or otherwise determines that it is to assist in completing in-body Digital Therapeutics or experiments. As another example, by contrast, the control system may initiate and complete steps 1621 through 1637, if it receives a request from a user, or otherwise determines that it is to assist in completing experiments involving an entire cohort of users of the control system, and/or mock experiments. Each of these example sets of steps will discussed in greater detail below.

Proceeding to step 1603, the control system may next, through, for example, techniques specified in this application, including but not limited to analyzing personal health-related data concerning a subject user, determine that a health-related event has occurred (such as an adverse health event, in some embodiments) for that user. Based on the occurrence, and time of occurrence, of that health-related event, the control system may next identify, create and/or form health-related aspects which it has monitored, which aspects have some correlation, or other linkage to the health-related event, in step 1605. In some embodiments, the control system does so by searching through previously-recorded health-related data, and establishing correlations between the occurrence of such aspects indicated in those data, and the health-related event. Next, the control system may determine which of those health-related aspects are most highly-correlated (or otherwise linked) with the health-related event, in step 1607. In some embodiments, the control system may next directly proceed to step 1611, in which it establishes a hypothesis that one or more of those highly-correlated (or otherwise linked) aspects is a cause of the health-related event.

However, in some embodiments, the control system first proceeds to intermediate step 1609, in which it performs a type of retroactive test or experiment, based on previously-recorded data. In some such embodiments, the control system may determine historical points in time, based on all data recorded in the control system relative to those aspects, whether those aspects were also highly correlated with the same (or similar) health-related events at those times. Based on positive results from such retroactive experiments, the control system may reaffirm, or discount, those aspects as potential causes of the health-related event, and instead focus on other variables in subsequent steps.

Next, the control system may proceed to formulate an in-body test or experiment, for example, by instigating particular data from the user in a new time period, in step 1613. In some embodiments, a user or the control system may set any number of variables, and control any number of other variables or factors, when creating the parameters for such in-body testing. For example, as discussed above, in some embodiments, such a test is carried out by instigating similar data as that suspected as indicating a cause, at a similar time of day as it occurred previously, as discussed above. In any event, such instigations of data are next carried out by the control system in step 1615. Following that instigation, and any waiting period for potential lag between the suspected cause and health-related event, the control system determines the results of the test or experiment—including, but not limited to, whether a similar health-related event has occurred—in step 1617.

The control system may repeat steps 1603 through 1617 any number of times, with respect to any number of health-related events or suspected causes of health-related events, as noted in subsequent step 1619. The control system then returns to the starting position.

Returning to the possibility of broader, population-wide studies, the control system also may initiate step 1621 to begin such studies, in which such a study may be authorized by the control system, or an administrative user with privileges to order the initiation of such population-wide studies (e.g., licensed medical professionals). Because such studies involve more than one user, data may be so restricted and anonymized and/or aggregated, and/or restricted to consenting users, in some embodiments, prior to using any such data in such studies. In some embodiments, all such data access, and grouping of user's data, is in strict compliance with applicable laws and jurisdictions in which the study is implemented.

The control system proceeds to step 1623, in which it may next identify, create and/or form health-related aspects which it has monitored, which relate, or potentially relate, to the subject of the study to be conducted by the control system. In some embodiments the control system next proceeds to step 1625, in which it designates a first historical time frame, in which at least some cohort of multiple users had data related to such aspects recorded in the past. In some embodiments, the control system assesses the amount of correlation between a subject result of interest of the study (e.g., reducing inflammation in arthritis or other auto-immune disorders) and such aspects. Next, in step 1627, hypotheses are generated, based in part on those correlations or other linkages. In some embodiments, the control system itself, using artificial intelligence techniques, assesses such correlations and linkages between aspects and results of interest in the study, and selects the most likely suspected causes of the results of interest as a hypothesis to be tested.

Next, and pursuant to retroactive experimental techniques set forth in this application, the control system may select a second historical time period, and/or a second cohort of users, for which similar data, for similar aspects, and under similar control variables indicated, have been recorded by the control system, in step 1629. If, in step 1631, the control system determines that a sufficiently large cohort (e.g., a number of users (N), yielding statistical significance based on the experimental parameters, controls, methods and/or error rate), and similar enough time period are available in such a second previous time period (T2), the control system proceeds to step 1633. (If not, in some embodiments, the control system returns to the starting position or, in other embodiments, awaits the creation of sufficient data in a time period emerging from databases managed by the control system.) In step 1633, the control system then runs the retroactive study, and determines if the hypothesis has been disproven, based on the result of implementing the aspect, under the same controlled circumstances, and testing whether the results of interest have not occurred.

In some embodiments, the results of the study may be validated by additional analysis and review, in step 1635. Also in some embodiments, the control system may then automatically publish an automatically-generated report (e.g., by sharing data and/or automatically-generated literature stating all tested results, cohort details, controls, and other methods and parameters of the study) to a peer-reviewed journal (e.g., via an Internet connection and publication platform), in step 1637.

Following any number of such sub-sets of steps, related to any number of aspects or effects for such tools or sub-tools, or other Digital Therapeutics, the control system may return to the starting position.

Of course, in a virtually unlimited number of alternative embodiments, a wide variety of alternative and/or additional steps or processes, fewer steps or processes, different orders of steps or processes, instances of steps or processes, arrangements of steps or processes, and other variations of the steps and/or processes, with additional or alternative timing and preconditions, may be provided, other than the examples specifically set in the present application, and such additional and alternative steps and processes also fall within the scope of the invention, as will be apparent to those of skill in the art. The exact steps, number of steps, sequences, orders of steps set forth herein are but one example, and do not limit the scope of the invention and disclosure. The examples set forth in the present application are merely examples, illustrating some principles of the invention.

What is claimed is:

1. A system for managing health-related data, comprising:
a control system comprising computer hardware and software configured to:
receive and record a first linguistic term entered by a user;
receive and record a plurality of other linguistic terms entered by the user and/or other users;
generate at least one algorithm based on relationships between the usage of at least one of said linguistic terms entered by a user and said other linguistic terms entered by the user and/or other users;
develop a new standardized meaning and assign a new, unique code for said meaning, and to associate said at least one of said linguistic terms with said code based, at least in part, on said algorithm;
store said health-related data related to said user, said new, unique code, and said meaning based on said new standardized meaning.

2. The system for managing health-related data of claim 1, wherein said control system comprising computer hardware and software is configured to:
match each of said linguistic terms with an existing standardized meaning developed by the control system, and at least one existing unique code for said meaning assigned by the control system.

3. The system for managing health-related data of claim 2, wherein said control system is configured to:
store health-related data related to said user, related to said existing standardized meaning and related to said existing unique code.

4. The system for managing health-related data of claim 1, wherein said new standardized meaning and new unique code are assigned to more than one of said linguistic terms entered by said user and/or said other users.

5. The system for managing health-related data of claim 1, wherein said new standardized meaning and new unique code are assigned to a plurality of terms of different human languages and/or dialects.

6. The system for managing health-related data of claim 1, wherein said at least one algorithm comprises a plurality of relationships.

7. The system for managing health-related data of claim 6, wherein said plurality of relationships are incorporated into a Significance Map.

8. The system for managing health-related data of claim 1, wherein said new standardized meaning and said a new, unique code are based on a Significance Map.

9. The system for managing health-related data of claim 8, wherein said health-related data is stored via a Translation Vector.

10. A method for managing health-related data, comprising the following steps:
providing a control system comprising computer hardware and software configured to:
receive and record a first linguistic term entered by a user;
receive and record a plurality of other linguistic terms entered by the user and/or other users;
generate at least one algorithm based on relationships between the usage of at least one of said linguistic terms entered by a user and said other linguistic terms entered by the user and/or other users;
develop a new standardized meaning and assign a new, unique code for said meaning, and to associate said at least one of said linguistic terms with said code based, at least in part, on said algorithm;
store said health-related data related to said user, said new, unique code, and said meaning based on said new standardized meaning.

11. The method for managing health-related data of claim 10, comprising the following additional steps:
receiving and recording a first linguistic term entered by a user.

12. The method for managing health-related data of claim 11, comprising the following additional steps:
receiving and recording a plurality of other linguistic terms entered by the user and/or other users.

13. The method for managing health-related data of claim 12, comprising the following additional step:
generating at least one algorithm based on relationships between the usage of at least one of said linguistic terms entered by a user and said other linguistic terms entered by the user and/or other users.

14. The method for managing health-related data of claim 13, comprising the following additional steps:
developing a new standardized meaning and assigning a new, unique code for said meaning, and associating said at least one of said linguistic terms with said code based, at least in part, on said algorithm.

15. The method for managing health-related data of claim 13, comprising the following additional step:
storing said health-related data related to said user, said new, unique code, and said meaning based on said new standardized meaning.

16. The method for managing health-related data of claim 10, comprising the following additional step:
providing a Significance Map to a user, comprising a line of meaning and a selectable sub-meanings related to said standardized meaning.
